(19) United States
(12) Patent Application Publication
(10) Pub. No.: US 2014/0218495 A1
(43) Pub. Date: Aug. 7, 2014

(54) MEDICAL IMAGE PHOTOGRAPHING SYSTEM AND MEDICAL IMAGE MANAGEMENT APPARATUS

(71) Applicant: KONICA MINOLTA, INC., Tokyo (JP)

(72) Inventor: Eiichi AKAGI, Tokyo (JP)

(73) Assignee: KONICA MINOLTA, INC., Tokyo (JP)

(21) Appl. No.: 14/171,717

(22) Filed: Feb. 3, 2014

(30) Foreign Application Priority Data
Feb. 4, 2013 (JP) 2013-019196

(51) Int. Cl. H04N 5/232

(52) U.S. Cl. CPC H04N 5/23222
USPC 348/77

(57) ABSTRACT

A medical image photographing system includes: a photographing device; a medical image management apparatus communicably connected thereto to manage the medical image; and a client terminal communicably connected thereto, wherein the client terminal includes: a display control section to causes the display section to display a list of photographing object information; a selection section to receive an selection input of one piece of photographing object information from the list with respect to the medical image; and a transmission section to transmit selection information, and the medical image management apparatus includes a correlation section to correlate the selected photographing object information with the medical image on the basis of the selection information to cause the storage section to store them, and the display control section causes the selected photographing object information to be displayed so that it is distinguishable from other photographing object information on the list.

### BACKGROUND OF THE INVENTION

1. Field of the Invention

The present invention relates to a medical image photographing system and a medical image management apparatus.

2. Description of Related Art

Heretofore, there has been used in a medical field a medical image management apparatus which fetches medical images generated in various photographing devices, and stores and manages the fetched medical images in a database. For example, there has been proposed a medical image management apparatus which stores a medical image transmitted from a photographing device while correlating it with patient information of a diagnostic target patient, and displays the medical image on a viewer screen, when an image fetch button provided on the viewer screen for the diagnostic target patient is pressed (for example, see Japanese Patent Application Laid-open No. 2010-124943).

Generally, a radiographer creates a list of portions to be photographed on the basis of a photographing order. The photographing order means a photographing request from a doctor, and includes request details regarding a series of photographing processes, such as “plain chest X-ray photography” and “lumbus four-direction photography”, which contains plural photographing operations. For example, in “plain chest X-ray photography”, two photographing operations of “front chest photography” and “side chest photography” are performed, and in “lumbus four-direction photography”, four photographing operations of “front lumbus photography”, “side lumbus photography”, “lumbus anteflextion photography” and “lumbus retroflection photography” are performed. When the radiographer selects a photographing portion from the list and performs photographing thereof, the medical image obtained by the photographing is correlated with the selected photographing portion on a one-to-one basis. In the case that photographing portions which have not been photographed yet remain in the list, the radiographer selects another photographing portion and repeats the processes similarly to the above. The examination is terminated when all photographing portions have been photographed.

However, if there is a large number of medical images to be taken in one examination, it is difficult for a radiographer to always remember whether or not all photographing portions have already been photographed, and there is a possibility that some portions are not mistakenly photographed. Especially in the examination of a racehorse, the number of images to be taken reaches thirty or more because the images are necessary for judging whether or not there are any defects such as fracture.

Moreover, in the case that a photographing object is an animal, unlike the case of human beings, stopping the motion of the photographing object by an instruction of a radiographer is difficult. For this reason, a plurality of photographing portions cannot always be photographed in a predetermined order, and it is necessary to flexibly change the photographing order as necessary. In such case, according to conventional techniques, it is necessary to select the photographing portion again in order to perform photographing.

For example, here is a case that a photographing object is racehorse and the following photographing portions/directions (1)-(8) are photographed.

(1) Right forefoot ankle joint: front face

(2) Right forefoot ankle joint: side face

(3) Left forefoot ankle joint: front face

(4) Left forefoot ankle joint: side face

(5) Right hind foot ankle joint: front face

(6) Right hind foot ankle joint: side face

(7) Left hind foot ankle joint: front face

(8) Left hind foot ankle joint: side face

After performing item (1) “right forefoot ankle joint; front face” first, it is efficient to subsequently perform item (2) “right forefoot ankle joint; side face” which targets the same right forefoot. However, photographing is sometimes continued from item (5) or subsequent items which target the hind feet when the horse suddenly starts to move his forefeet. In such case, some selection change operations become necessary because the photographing portion and direction need to be selected before performing photographing. However, if the horse starts to move his hind feet during such operations, it becomes impossible to photograph the hind feet. Thus, the conventional operations for changing photographing order are troublesome.

### SUMMARY OF THE INVENTION

The present invention is made in view of the foregoing problems of conventional techniques, and an object of the present invention is to prevent omission of photographing and to make photographing in an arbitrary order easy when photographing a plurality of photographing portions.

In order to achieve the above object, according to a first aspect of the present invention, there is provided a medical image photographing system including: a photographing device that generates at least one medical image of a photographing object; a medical image management apparatus that is connected to the photographing device so as to enable data communication with the photographing device and manages the medical image generated by the photographing device; and a client terminal that is connected to the medical image management apparatus so as to enable data communication with the medical image management apparatus, wherein the client terminal includes: a display section: a display control section to cause the display section to display, on the basis of a plurality of pieces of photographing object information including at least a photographing portion, a list of the photographing object information; a selection section to receive an input for selecting any one piece of photographing object information, from the list displayed in the display section, with respect to the medical image generated by the photographing device; and a transmission section to transmit selection information indicating the selected photographing object information to the medical image management apparatus, and the medical image management apparatus includes: a storage section; and a correlation section to correlate the selected photographing object information with the medical image generated by the photographing device on the basis of the selection information transmitted from the client terminal to cause the storage section to store the selected photographing object information correlated to the medical image, and the display control section causes the selected photographing object information to be displayed, on the list displayed in the display section, so that the selected photographing object information is distinguishable from other photographing object information.

By the first aspect of the present invention, because the selected photographing object information is displayed so that it is distinguishable from other photographing object information on the list, the photographing object information which has not been correlated with the medical image can be easily recognized when taking images of plural photographing portions, which can prevent omission of photographing. Moreover, when photographing the plural photographing portions, photographing in an arbitrary order can be easily performed because the photographing object information is correlated to the medical image created by the photographing device.

Preferably, in the medical image photographing system, the correlation section is capable of correlating the same photographing object information with the plurality of medical images generated by the photographing device.

Preferably, in the medical image photographing system, the display control section causes the selected photographing object information to be displayed, on the list displayed in the display section, so that the selected photographing object information is distinguishable from other photographing object information, by adding a mark, a character, a background color, or the medical image corresponding to the selected object information.

Preferably, in the medical image photographing system, the display control section causes the selected photographing object information to be displayed, on the list displayed in the display section, so that the selected photographing object information is distinguishable from other photographing object information, by changing a display position of the selected photographing object information into a different position from a display position of the other photographing object information.

Preferably, in the medical image photographing system, the photographing object information further includes a photographing direction or a body posture.

Preferably, in the medical image photographing system, the display control section causes a photographing condition corresponding to each piece of the photographing object information to be displayed, on the list displayed in the display section, in addition to each piece of the photographing object information.

According to a second aspect of the present invention, there is provided a medical image management apparatus which is connected to a photographing device that generates at least one medical image of a photographing object so as to enable data communication with the photographing device, and manages the medical image generated by the photographing device, the medical image management apparatus including: a display section: a display control section to cause the display section to display, on the basis of a plurality of pieces of photographing object information including at least a photographing portion, a list of the photographing object information; a selection section to receive an input for selecting any one piece of photographing object information, from the list displayed in the display section, with respect to the medical image generated by the photographing device; a storage section; and a correlation section to correlate the selected photographing object information with the medical image generated by the photographing device to store the selected photographing object information correlated to the medical image, wherein the display control section causes the selected photographing object information to be displayed, on the list displayed in the display section, so that the selected photographing object information is distinguishable from other photographing object information.

By the second aspect of the present invention, because the selected photographing object information is displayed so that it is distinguishable from other photographing object information on the list, the photographing object information which has not been correlated with the medical image can be easily recognized when taking images of plural photographing portions, which can prevent omission of photographing. Moreover, when photographing the plural photographing portions, photographing in an arbitrary order can be easily performed because the photographing object information is correlated to the medical image created by the photographing device.

Preferably, in the medical image photographing system, the correlation section is capable of correlating the same photographing object information with the plurality of medical images generated by the photographing device.

Preferably, in the medical image photographing system, the display control section causes the selected photographing object information to be displayed, on the list displayed in the display section, so that the selected photographing object information is distinguishable from other photographing object information, by adding a mark, a character, a background color, or the medical image corresponding to the selected object information.

Preferably, in the medical image photographing system, the display control section causes the selected photographing object information to be displayed, on the list displayed in the display section, so that the selected photographing object information is distinguishable from other photographing object information, by changing a display position of the selected photographing object information into a different position from a display position of the other photographing object information.

Preferably, in the medical image photographing system, the photographing object information further includes a photographing direction or a body posture.

Preferably, in the medical image photographing system, the display control section causes a photographing condition corresponding to each piece of the photographing object information to be displayed, on the list displayed in the display section, in addition to each piece of the photographing object information.

### BRIEF DESCRIPTION OF THE DRAWINGS

The present invention will become more fully understood from the detailed description given hereinbelow and the appended drawings, and thus are not intended as a definition of the limits of the present invention, and wherein:

FIG. 1 is a system configuration diagram of a medical image photographing system according to a first embodiment;

FIG. 2 is a perspective view illustrating an exterior appearance of an FPD panel;

FIG. 3 is a block diagram illustrating an equivalent circuit of the FPD panel;

FIG. 4 is a block diagram illustrating a functional configuration of a medical image management apparatus and a client terminal;

FIG. 5 illustrates an example of a photographing object information table;

FIG. 6 illustrates an example of an image information table;

FIG. 7 is a ladder diagram illustrating medical image photographing processing in the medical image photographing system;

FIG. 8 is a ladder diagram illustrating medical image photographing processing in the medical image photographing system;

FIG. 9 is a ladder diagram illustrating medical image photographing processing in the medical image photographing system;

FIG. 10 is a diagram illustrating an example of an image display screen;

FIG. 11 is a diagram illustrating an example of an image display screen;

FIG. 12 is a diagram illustrating an example of an image display screen;

FIG. 13 is a diagram illustrating an example of an image display screen according to a second embodiment;

FIG. 14 is a diagram illustrating an example of an image display screen according to a third embodiment;

FIG. 15 is a diagram illustrating an example of an image display screen according to a fourth embodiment;

FIG. 16 is a diagram illustrating an example of an image display screen according to a fifth embodiment;

FIG. 17 is a block diagram illustrating a functional configuration of a medical image management apparatus; and

FIG. 18 is a flowchart illustrating processing in the medical image management apparatus.

### PREFERRED EMBODIMENT OF THE PRESENT INVENTION

### First Embodiment

Hereinafter, a first embodiment of a medical image photographing system of the present invention will be described with reference to the drawings. In this regard, however, the scope of the present invention is not limited to illustrated examples.

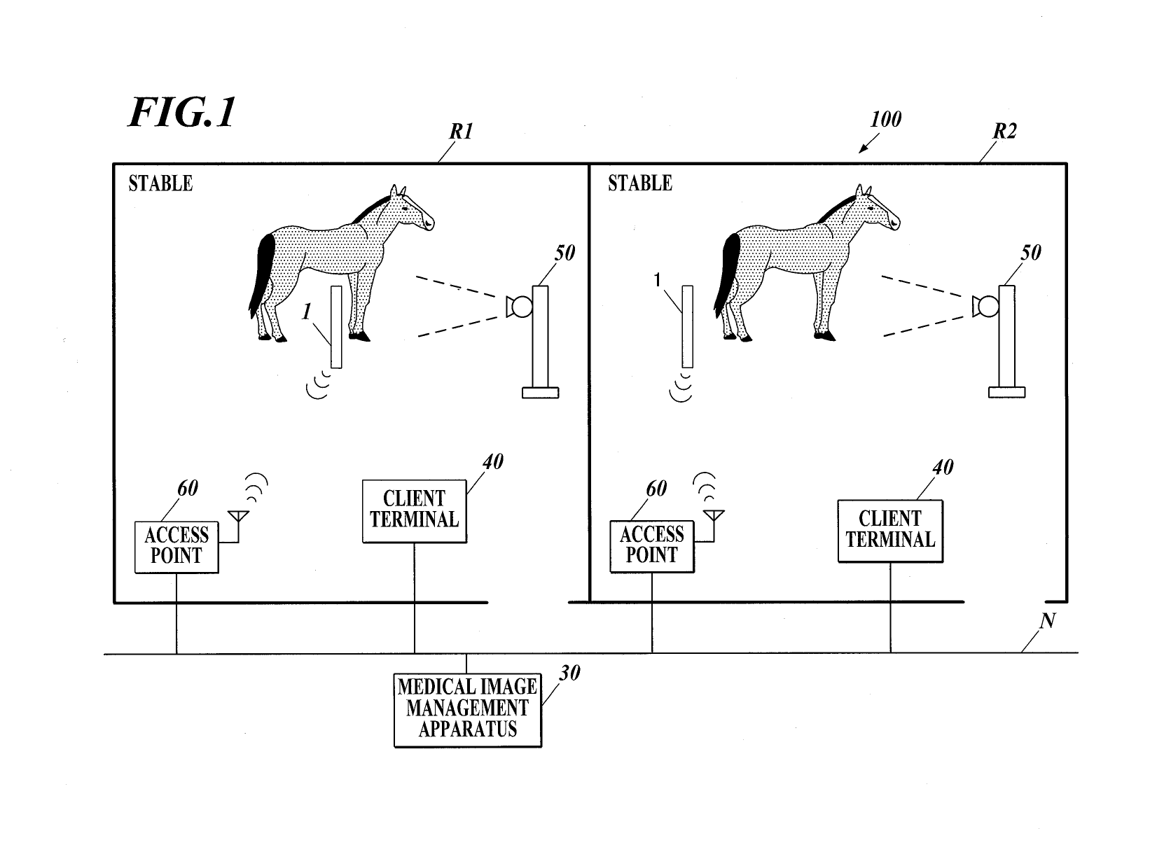

FIG. 1 illustrates a system configuration diagram of the medical image photographing system 100 according to the first embodiment. The medical image photographing system 100 is configured to include a Flat Panel Detector (FPD) panel 1, a medical image management apparatus 30, a client terminal 40, a radiation generation apparatus 50, an access point 60, and so on. The medical image management apparatus 30 and the client terminal 40 are connected to each other via a communication network N so that data communication can be performed therebetween. The FPD panel 1 and the medical image management apparatus 30 are connected to each other via the access point 60 and the communication network N so that data communication can be performed therebetween.

There are provided the FPD panel 1, the client terminal 40, the radiation generation apparatus 50 and the access point 60 in each of stables R1, R2. In the stables R1, R2, radiation images of racehorses as photographing objects are taken.

The FPD panel 1 is a flat cassette type photographing device which generates medical images of photographing objects. The FPD panel 1 creates image data of radiation images on the basis of radiation which has been emitted from the radiation generation apparatus 50 and has transmitted through objects. The FPD panel 1 detects irradiation timing of radiation emitted from the radiation generation apparatus 50 and generates the medical images depending on the radiation irradiation.

The radiation generation apparatus 50 emits a predetermined amount of radiation for a predetermined time, on the basis of radiation irradiation conditions which have been set, when a switch for inputting instructions as to irradiation timing is pressed.

The access point 60 is provided with an antenna for performing wireless data communication with the FPD panel 1.

The medical image management apparatus 30 manages the medical images generated by the photographing device such as the FPD panel 1 and information as to horses while correlating them with each other, and provides the medical images and the information of horses in response to requests from external devices/apparatus. The information of horses is equivalent to patient information of human beings, and include a horse identification ID, a name of horse, gender, birth date, and so on. The medical image management apparatus 30 can be installed in any locations where it can connect to the communication network N.

The client terminal 40 is a computer apparatus for instructing the medical image management apparatus 30 to acquire the medical images from the photographing device such as the FPD panel 1, and/or for instructing the medical image management apparatus 30 to refer to the medical images managed therein.

Although illustration is omitted, a Hospital Information System (HIS), a Radiology Information System (RIS), other computers, and/or an imager which records radiation images in an image recoding medium such as a film and outputs them, are connected to the communication network N.

Next, a configuration of the FPD panel 1 will be described.

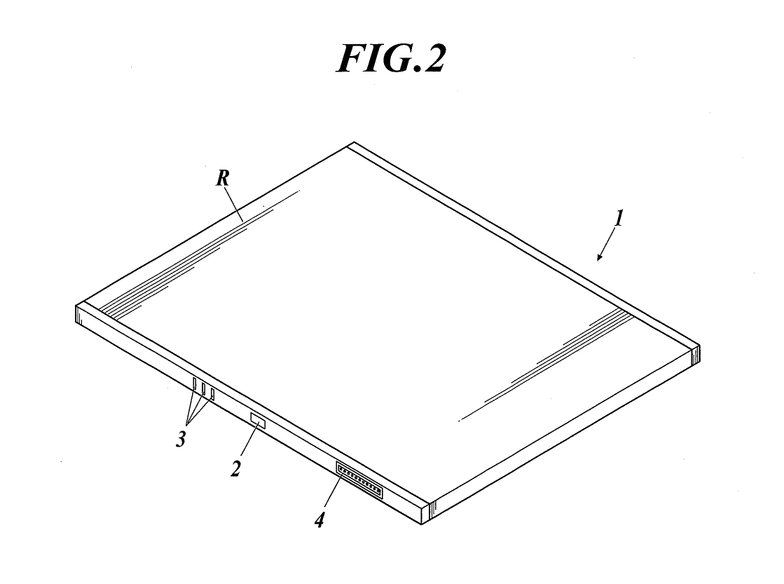

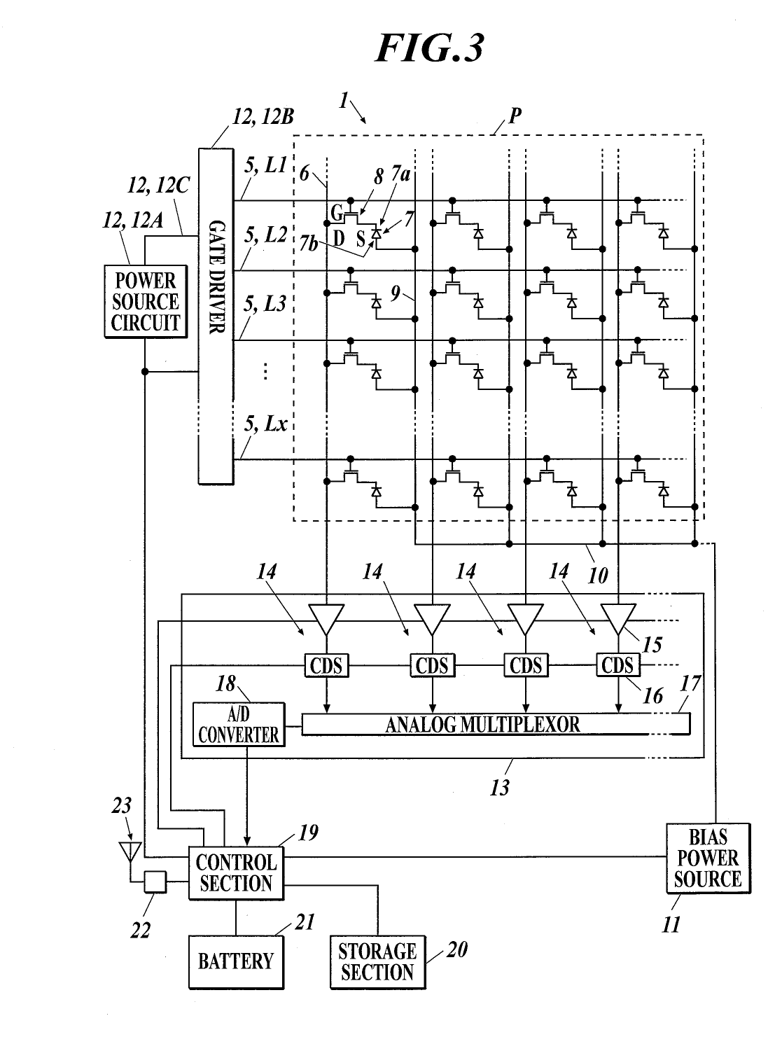

FIG. 2 is a perspective view illustrating an exterior appearance of the FPD panel 1. As illustrated in FIG. 2, the FPD panel 1 is configured to be equipped with a housing including a radiation incident surface R which is a surface irradiated with radiation. The housing includes: a power switch 2 for turning on/off a power source of the FPD panel 1; an indicator 3 which is composed of a Light Emitting Diode (LED) or the like to indicate a state of a battery 21 (see FIG. 3) and/or an operation state of the FPD panel 1, etc.; and a connector 4 to be used for charging the battery 21. Although illustration is omitted in FIG. 2, the FPD panel 1 is also equipped with an antenna device 23 (see FIG. 3) for performing wireless communication with an external device/apparatus such as the medical image management apparatus 30 through the access point 60.

FIG. 3 is a block diagram illustrating an equivalent circuit of the FPD panel 1. As illustrated in FIG. 3, a plurality of scanning lines 5 and signal lines 6 are arranged so as to intersect with each other in a detection section P of the FPD panel 1. In each of small regions divided by the scanning lines 5 and the signal lines 6, a radiation detection element 7 is provided. Thus, a plurality of radiation detection elements 7 are two-dimensionally arranged in a matrix. Although a photo diode is used as the radiation detection element 7 in this embodiment, for example, other radiation detection elements such as a phototransistor may be used.

To a first electrode 7A of each radiation detection element 7, a source electrode 8S (“S” in FIG. 3) of a Thin Film Transistor (TFT) 8 as a switching member is connected. A drain electrode 8D (“D” in FIG. 3) of the TFT 8 is connected to each signal line 6, and a gate electrode 8G (“G” of FIG. 3) of the TFT 8 is connected to each scanning line 5.

The TFT 8 is turned on when an ON voltage is applied to the gate electrode 8G from a scanning driving member 12 via each scanning line 5, and causes electrical charge accumulated in the radiation detection element 7 to be discharged to each signal line 6 via the source electrode 8S and/or the drain electrode 8D. The TFT 8 is turned off when an off voltage is applied to the gate electrode 8G via each scanning line 5 connected thereto, and stops discharge of the electrical charge from the radiation detection element 7 to each signal line 6 so that electrical charge is accumulated in the radiation detection element 7.

Bias lines 9 are provided so that each bias line 9 corresponds to the radiation detection elements 7 contained in each column, namely the radiation detection elements 7 arranged in a longitudinal direction in FIG. 3. Each second electrode 7B of each radiation detection element 7 is connected to each bias line 9. The bias lines 9 are connected to a tie line 10 outside the detection section P. The tie line 10 is connected to a bias power source 11 so that the bias power source 11 applies opposite bias voltages to the second electrodes 7B of the radiation detection elements 7 via the tie line 10 and the bias lines 9.

The scanning lines 5 are connected to a gate driver 12B of the scanning driving member 12. In the scanning driving member 12, ON voltage and OFF voltage are supplied from a power source circuit 12A to the gate driver 12B via a wiring 12C. The gate driver 12B switches a voltage to be applied to each of lines L1 to Lx of scanning lines between ON voltage and OFF voltage so as to control on/off operation of each of the TFTs 8.

Each of the signal lines 6 is connected to each of reading circuits 14 incorporated in a reading IC 13. Each of the reading circuits 14 is composed of an amplifier circuit 15, a correlated double sampling (CDS) circuit 16, etc. The reading IC 13 further includes an analog multiplexor 17, an A/D converter 18, etc.

In processing of reading out image data from each radiation detection element 7, when ON voltage is applied to a predetermined line Ln among the scanning lines 5 from the gate driver 12B of the scanning driving member 12, ON voltage is applied each gate electrode 8G of the TFT 8 connected to the predetermined line Ln via the line Ln among the scanning lines 5, the TFTs 8 is turned on, and electrical charge is discharged from the radiation detection elements 7 respectively connected to the TFTs 8 in ON state to the signal lines 6 via the TFTs 8.

Each amplifier circuit 15 outputs a voltage value depending on an amount of electrical charge, which has flowed thereinto via each signal line 6, from an output side of the amplifier 15. Each correlated double sampling circuit 16 outputs an increased amount between output values from the amplifier circuit 15 before and after the electrical charge flows thereinto from the radiation detection element 7, as image data of analog values, to the downstream of the correlated double sampling circuit 16.

The output image data are then sequentially transmitted to the A/D converter 18 via the analog multiplexor 17, sequentially converted to image data of digital values in the A/D converter 18, and output to a storage section 20 and sequentially stored therein.

A control section 19 is composed of a not-illustrated Central Processing Unit (CPU), a Read Only Memory (ROM), a Random Access Memory (RAM), a computer including an input/output interface etc. connected to a bus, a Field Programmable Gate Array (FPGA), and so on. Incidentally, the control section 19 may be composed of a dedicated control circuit.

The control section 19 controls operations of the respective functional sections of the FPD panel 1, for example, controls the scanning driving member 12 and/or the reading circuits 14 to perform image data reading processing. To the control section 19, the storage section 20 which is composed of a Static RAM (SRAM), a Synchronous DRAM (SDRAM), and the like is connected.

Moreover, also a battery 21 which supplies electrical power necessary for the respective functional sections such as the scanning driving member 12, the reading circuit 14, the storage section 20, and the bias power source 11 is connected to the control section 19.

Furthermore, also a wireless communication section 22 is connected to the control section 19, which performs wireless communication with an external device/apparatus via the antenna device 23. The wireless communication section 22 is configured to perform wireless communication with an external device/apparatus by using, for example, an IEEE 802.11 wireless LAN.

The FPD panel 1 can be turned into one of three-stage states of “driving state”, “driving ready state” and “non-driving state”.

The driving state is a state that the FPD panel 1 can perform photographing anytime. Concretely, the driving state is a state where the gate driver 12B applies ON voltage to the lines L1 to Lx of the scanning lines 5 to repeatedly perform reading out of electrical charge accumulated in the radiation detection element 7 so that the electrical charge is always reset. The FPD panel 1 can start accumulation of electrical charge anytime by applying OFF voltage to the gate electrodes 8G via the scanning lines 5 by the gate driver 12B. When the FPD panel 1 is in the driving state, power consumption is largest among those in the three-stage states.

The driving ready state is a state that the FPD panel 1 can promptly transfer to the driving state. The amount of time for transferring from the driving ready state to the driving state is shorter than the amount of time for transferring from the non-driving state to the driving state. Concretely, the driving ready state is a state where ON/OFF voltages are not applied to the lines L1 to Lx of the scanning lines 5 and reading out is stopped. The others are same as those of the driving state. The amount of power consumption is less than the driving state by the electrical power for reading out electrical charge. Thus, when the FPD panel 1 is in the driving ready state, power consumption is less than that in the driving state, but larger than that in the non-driving state. Although a user cannot promptly perform photographing if he/she tries to do so in the non-driving state because it takes a few seconds for transferring from the non-driving state to the driving state, a user can transfer to the driving state anytime by reading out electrical charge accumulated in the radiation detection element 7 so that the electrical charge is reset in the driving ready state.

The non-driving state is a state that power supply to at least a part of the FPD panel 1 is stopped and photographing cannot be performed. Concretely, the non-driving state is a state where electrical power is supplied to only functional sections such as the wireless communication section 22, the control section 19, etc. which need to be activated at minimum so as to receive signals from an external device/apparatus, and power supply to other sections is stopped. When the FPD panel 1 is in the non-driving state, power consumption is smallest among those in the three-stage states.

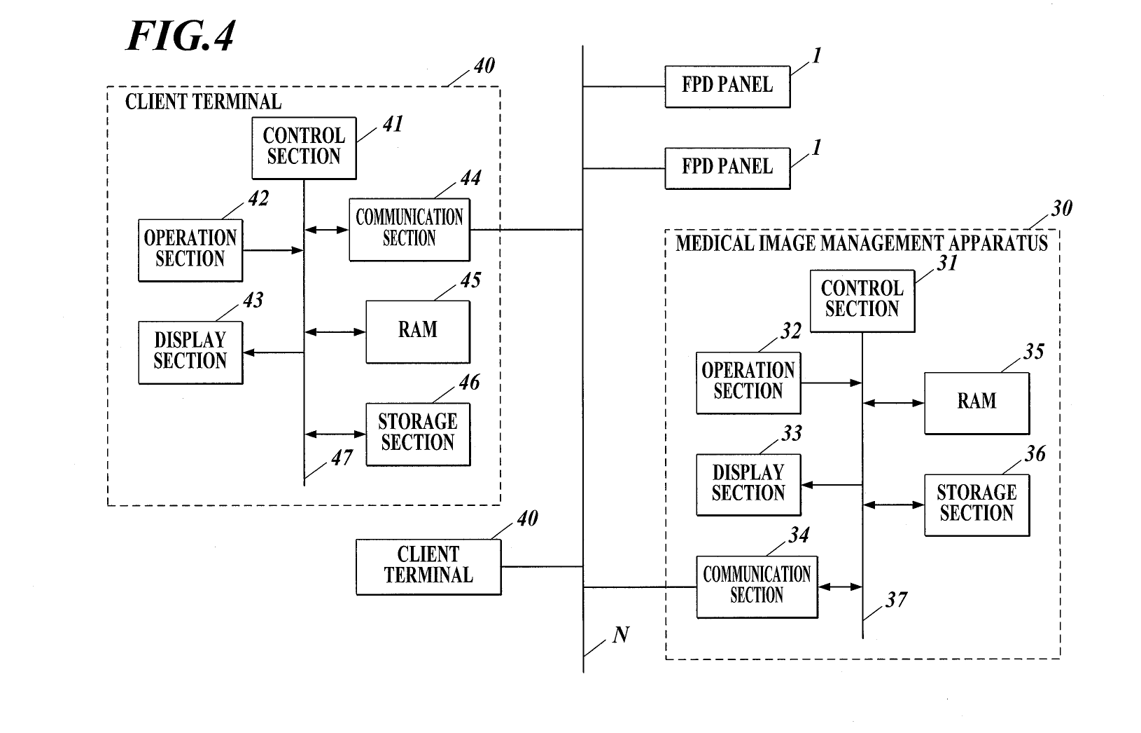

Next, the functional configurations of the medical image management apparatus 30 and the client terminal 40 will be described with reference to FIG. 4.

As illustrated in FIG. 4, the client terminal 40 is configured to include a control section 41, an operation section 42, a display section 43, a communication section 44, a RAM 45, a storage section 46, etc., and the respective sections are connected to via a bus 47 one another.

The control section 41 is composed of a CPU and the like, reads out various programs such as system programs and processing programs stored in the storage section 46 to expand them in the RAM 45, and executes various processes according to the expanded programs.

The operation section 42 is configured to include a keyboard equipped with character input keys, numeric input keys, various functional keys, etc., a pointing device such as a mouse, and so on, and outputs a pressing signal of each key which has been pressed on the keyboard and an operation signal from the mouse to the control section 41 as input signals.

The operation section 42 is used for selecting any one piece of photographing object information from a list displayed in the display section 43, with respect to the medical image generated in the photographing device such as the FPD panel 1. In other words, the operation section 42 functions as a selection section.

The photographing object information is information indicating which portion of the photographing object is to be photographed, and includes at least a photographing portion. The photographing portion indicates a portion to be photographed, such as a right forefoot ankle joint. The photographing object information further includes a photographing direction and/or a body posture. The photographing direction is information indicating a direction/orientation, such as a front face and side face, of the photographing portion when performing photographing. The photographing direction may include information indicating a direction of photographing such as AP and PA. The body posture is information indicating a position/posture of a body such as a spine position (lie face up), a lateral position (face sideways), etc. This embodiment describes the case that the photographing object information includes the photographing portions and the photographing directions.

The display section 43 is composed of a Liquid Crystal Display (LCD), and displays various screens in accordance with instructions of display signals input from the control section 41. For example, the medical images transmitted from the medical image management apparatus 30 are displayed in the display section 43.

The communication section 44 is composed of a network interface and the like, and performs data transmission/reception with an external device/apparatus connected to the communication network N. For example, the communication section 44 transmits selection information indicating selected photographing object information to the medical image management apparatus 30. Thus, the communication section 44 functions as a transmission section.

The communication section 44 may perform data transmission/reception with the external device/apparatus connected to the communication network N by wireless communication such as Wireless Fidelity (Wi-Fi). For example, the communication section 44 is implemented by using an antenna, RF converter, etc. for transmitting/receiving wireless signals via a wireless base station installed by a communication service provider. Alternatively, the communication section 44 may perform wireless communication with the medical image management apparatus 30 by infrared communication (IrDA/IrMC), Bluetooth (registered trademark), or the like.

The RAM 45 functions as a work area in which various programs read out from the storage section 46 and executable by the control section 41, input/output data, etc. are temporarily stored in various processes executed and controlled by the control section 41.

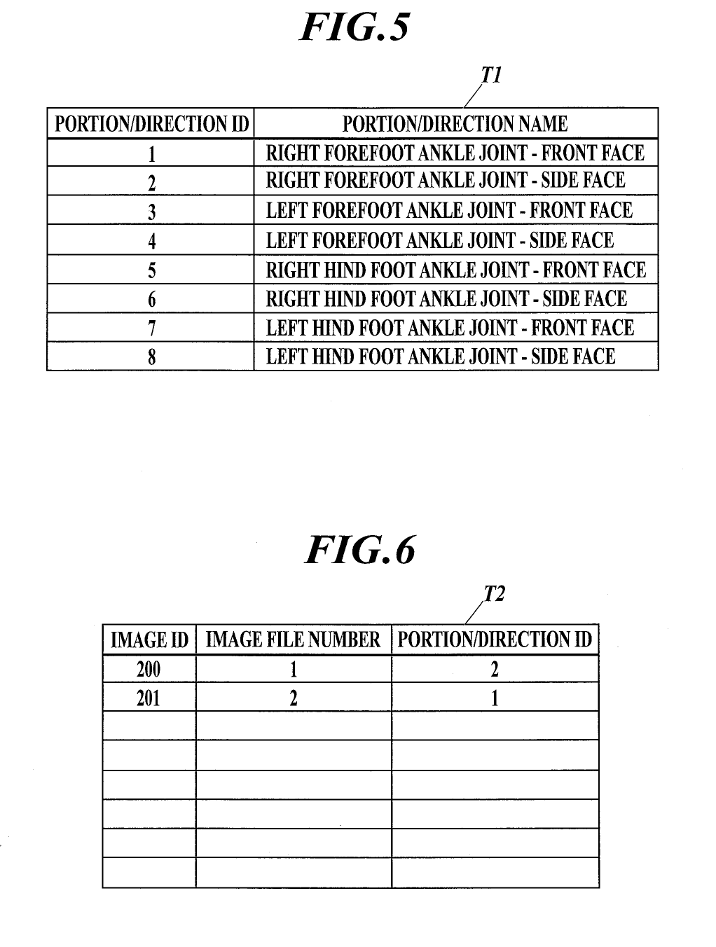

The storage section 46 is composed of a Hard Disk Drive (HDD), a semiconductor nonvolatile memory, or the like. The storage section 46 stores various programs, data necessary for various processes, etc. For example, the storage section 46 stores a photographing object information table T1 (see FIG. 5).

FIG. 5 illustrates an example of the photographing object information table T1.

The photographing object information table T1 is composed of a plurality of pieces of photographing object information, and a portion/direction ID and a portion/direction name are correlated to each piece of the photographing object information. The portion/direction ID is identification information given in order to identify each combination of the photographing portion and the photographing direction. For example, portion/direction ID “1” is correlated to the portion/direction name “right forefoot ankle joint: front face”. The photographing object information table T1 is previously prepared for each of plural examinations registered as racehorse examinations.

The control section 41 causes the display section 43 to display a list (portion/direction list) of photographing object information included in the photographing object information table T1, on the basis of the photographing object information table T1 containing the plural pieces of photographing object information. Thus, the control section 41 functions as a display control section.

The control section 41 controls the communication section 44 to transmit the selection information indicating the selected photographing object information to the medical image management apparatus 30.

The control section 41 causes the selected photographing object information to be displayed so that it is distinguishable from other photographing object information on the portion/direction list displayed in the display section 43, on the basis of an image information table T2 (see FIG. 6) stored in the storage section 36 of the medical image management apparatus 30.

Concretely, the control section 41 causes the selected photographing object information to be displayed so that it is distinct from other photographing object information on the portion/direction list displayed in the display section 43 by adding a check mark thereto.

As illustrated in FIG. 4, the medical image management apparatus 30 is configured to include a control section 31, an operation section 32, a display section 33, a communication section 34, a RAM 35, a storage section 36, etc., which are connected to one another by a bus 37.

The control section 31 is composed of a CPU and the like, and reads out various programs such as system programs and processing programs stored in the storage section 36 to expand them in the RAM 35, and executes various processes according to the expanded programs.

The operation section 32 is configured to include a keyboard equipped with character input keys, numeric input keys, various functional keys, etc., a pointing device such as a mouse, and so on, and outputs a pressing signal of each key which has been pressed on the keyboard and an operation signal from the mouse to the control section 41 as input signals.

The display section 33 is configured to include a monitor such as an LCD, and displays various screens in accordance with display signals input from the control section 31.

The communication section 34 is composed of a network interface or the like, and performs data transmission/reception with an external device/apparatus connected to the communication network N.

The RAM 35 functions as a work area in which various programs read out from the storage section 36 and executable by the control section 31, input/output data, etc. are temporarily stored in various processes executed and controlled by the control section 31.

The storage section 36 is composed of a Hard Disk Drive (HDD), a semiconductor nonvolatile memory, or the like. The storage section 36 stores various programs, data necessary for various processes, etc. For example, the storage section 36 stores the image information table T2 (see FIG. 6).

FIG. 6 illustrates an example of the image information table T2.

The image information table T2 is a table in which each piece of selected photographing object information is correlated with each medical image generated by the photographing device such as the FPD panel 1. Concretely, each portion/direction ID is correlated with each image ID and image file number in the image information table T2. The image ID is identification information given in order to identify each of the medical images. The image file number is a number indicating each of the medical image files. The image information table T2 is created for each horse to be photographed and for each examination.

The control section 31 correlates the selected photographing object information with the medical image generated by the photographing device such as the FPD panel 1 on the basis of the selection information transmitted from the client terminal 40, and causes the storage section 36 to store the selected photographing object information correlated to the medical image. Thus, the control section 31 functions as a correlation section.

The control section 31 may correlate the same photographing object information with the plural medical images generated by the photographing device such as the FPD panel 1.

Next, an operation of the medical image photographing system 100 will be described.

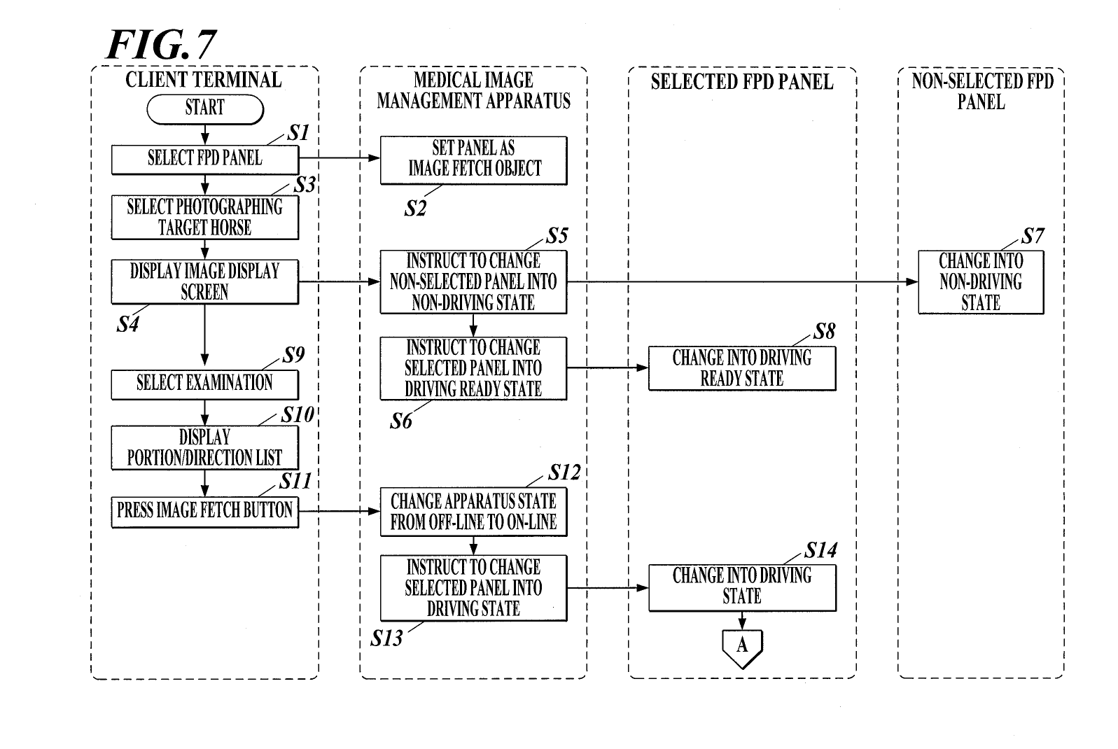

FIGS. 7 to 9 are ladder diagrams illustrating medical image photographing processing in the medical image photographing system 100. Here, the case where a horse is photographed in the stable R1 will be described as an example.

First, in the client terminal 40 installed in the stable R1, the control section 41 obtains information indicating each FPD panel 1 in the medical image photographing system 100 and information as to horses registered in the medical image management apparatus 30 from the medical image management apparatus 30 via the communication section 44, and causes the display section 43 to display a reception screen on the basis of the obtained information. The reception screen contains a panel selection region and a horse list display region. The panel selection region includes panel selection buttons for selecting each FPD panel 1 in the medical image photographing system 100. The horse list display region includes a list of horses.

When any one of the panel selection buttons in the panel selection region is pressed by operation using the operation section 42 and the FPD panel 1 in the stable R1 as an image fetching target is selected (Step S1), the control section 41 transmits the information indicating the selected FPD panel 1 to the medical image management apparatus 30 via the communication section 44.

In the medical image management apparatus 30, the communication section 34 receives the information indicating the selected FPD panel 1, and the control section 31 sets the selected FPD panel 1 as the FPD panel 1 which is of the image fetching target (Step S2).

Next, in the client terminal 40, when a photographing target horse is selected among horses displayed in the horse list display region on the reception screen by operation using the operation section 42 (Step S3), the control section 41 transmits the information indicating the selected photographing target horse to the medical image management apparatus 30 via the communication section 44, and causes the display section 43 to display an image display screen for displaying the image of the photographing target horse (Step S4).

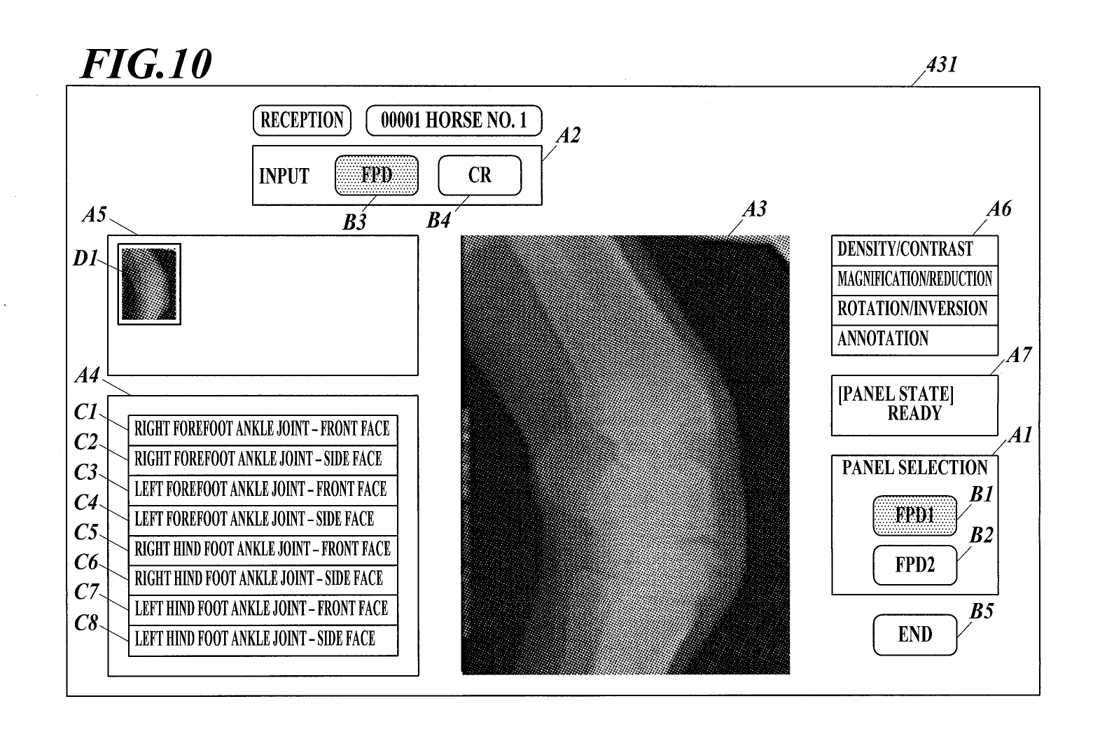

FIG. 10 illustrates an example of the image display screen 431.

The image display screen 431 includes a panel selection region A1, an image fetching instruction region A2, an image display region A3, a list display region A4, a photographed image display region A5, an image editing region A6, and a panel state display region A7.

The panel section region A1 contains panel selection buttons B1, B2. The panel section button B1 corresponds to the FPD panel 1 in the stable R1, and the panel selection button B2 corresponds to the FPD panel 1 in the stable R2. Since the FPD panel 1 in the stable R1 is selected in Step S1, the panel selection button B1 is in a pressed state in FIG. 10.

The image fetching instruction region A2 includes image fetch buttons B3, B4. Each of the image fetch buttons B3, B4 is a button for instructing to fetch the medical image transmitted from the corresponding photographing device as the image of a current diagnosis target horse. The image fetch button B3 corresponds to the FPD panels 1 in the stables R1, R2, and the image fetch button B4 corresponds to a not-illustrated Computed Radiography (CR) device. The image fetch buttons B3, B4 function, while being in the pressed states, as image fetch cancelling buttons for cancelling an image fetch ready state in the medical image management apparatus 30.

The image display region A3 is a region in which the medical image generated by the photographing device such as the FPD panel 1 and taken in the medical image management apparatus 30, or the medical image corresponding to a thumbnail image selected in the photographed image display region A5, is displayed. Incidentally, no image is displayed in the image display region A3 before fetching the image and/or before selecting the thumbnail image.

The list display region A4 is a region where a list of pieces of photographing object information, namely, a list of combinations of the photographing portion and the photographing direction to be used in the examination to be conducted. Incidentally, the list display region A4 is blank before the examination is selected.

The photographed image display region A5 is a region in which thumbnail images of taken medical images are displayed.

The image editing region A6 is used when performing density/contrast adjustment, magnification/reduction processing, rotation/inversion processing, adding of annotation, etc.

The panel state display region A7 is a region where the panel state of the selected FPD panel 1 is displayed.

In the medical image management apparatus 30, when the communication section 34 receives the information indicating the selected photographing target horse, namely, at the timing when the screen displayed in the display section 43 of the client terminal 40 changes into the image display screen (the screen on which medical image fetch can be instructed) of the photographing target horse, the control section 31 transmits a non-driving instruction for instructing the non-selected FPD panel 1 (the FPD panel 1 in the stable R2) among the plural FPD panels to shift to a non-driving state via the communication section 34 (Step S5), and transmits a driving ready instruction for instructing the selected FPD panel 1 (the FPD panel in the stable R1) to shift to a driving ready state via the communication section 34 (Step S6).

In the non-selected FPD panel 1 (the FPD panel 1 in the stable R2), when the wireless communication section 22 receives the non-driving instruction, the control section 19 changes the non-selected FPD panel 1 into the non-driving state (Step S7).

In the selected FPD panel 1 (the FPD panel 1 in the stable R1), when the wireless communication section 22 receives the driving ready instruction, the control section 19 changes the selected FPD panel 1 into the driving ready state (Step S8).

Next, in the client terminal 40 installed in the stable R1, when the examination to be performed this time is selected among the plural examinations previously registered as racehorse examinations by operation using the operation section 42 (Step S9), the control section 41 reads out the photographing object information table T1 corresponding to the selected examination from the storage section 46. Then, the control section 41 causes the display section 43 to display the list (portion/direction list) of combinations of photographing portions and photographing directions to be used in the selected examination on the basis of the plural pieces of photographing object information contained in the photographing object information table T1 which has been read out (Step S10).

For example, as illustrated in FIG. 10, the portion/direction list is displayed in the list display region A4 on the image display screen 431. Here, as the combinations of the photographing portions and the photographing directions included in the selected examination, “right forefoot ankle joint: front face” C1, “right forefoot ankle joint: side face” C2, “left forefoot ankle joint: front face” C3, “left forefoot ankle joint: side face” C4, “right hind foot ankle joint: front face” C5, “right hind foot ankle joint: side face” C6, “left hind foot ankle joint: front face” C7, and “left hind foot ankle joint: side face” C8 are displayed.

Incidentally, as the photographing object information table T1, not only using the one previously stored in the storage section 46, but also creating such table on the basis of the photographing object information is possible.

Next, in the client terminal 40 installed in the stable R1, when the image fetch button on the image display screen is pressed by operation using the operation section 42 (Step S11), the control section 41 transmits the instruction (image fetching instruction) to fetch the medical image transmitted from the photographing device corresponding to the pressed image fetch button, as the image of the photographing target horse, to the medical image management apparatus 30 via the communication section 44.

For example, when the image fetch button B3 is pressed on the image display screen 431 illustrated in FIG. 10, the control section 41 transmits the instruction to fetch the medical image transmitted from the FPD panel 1 selected in the panel selection region A1, to the medical image management apparatus 30.

In the medical image management apparatus 30, when the communication section 34 receives the image fetching instruction, the control section 31 changes the apparatus state from the off-line state to the on-line state (Step S12). Concretely, the control section 31 holds the information for specifying the client terminal 40 which has transmitted the image fetching instruction, and turns the medical image management apparatus 30 into the ready state for fetching the medical image created in the selected FPD panel 1. Incidentally, the medical image management apparatus 30 is configured so as not to receive image fetching instructions transmitted from other client terminals 40 until it receives the instruction to cancel the image fetching ready state from the client terminal 40 which has transmitted the image fetching instruction.

Moreover, in the medical image management apparatus 30, when the communication section 34 receives the image fetching instruction, the control section 31 transmits the driving instruction that instructs the selected FPD panel 1 (the FPD panel 1 in the stable R1) to shift to the driving state via the communication section 34 (Step S13). The control section 31 also sets the client terminal 40 (the client terminal 40 in the stable R1) which has transmitted the image fetching instruction as the destination to which the medical image created by the FPD panel 1 is transmitted.

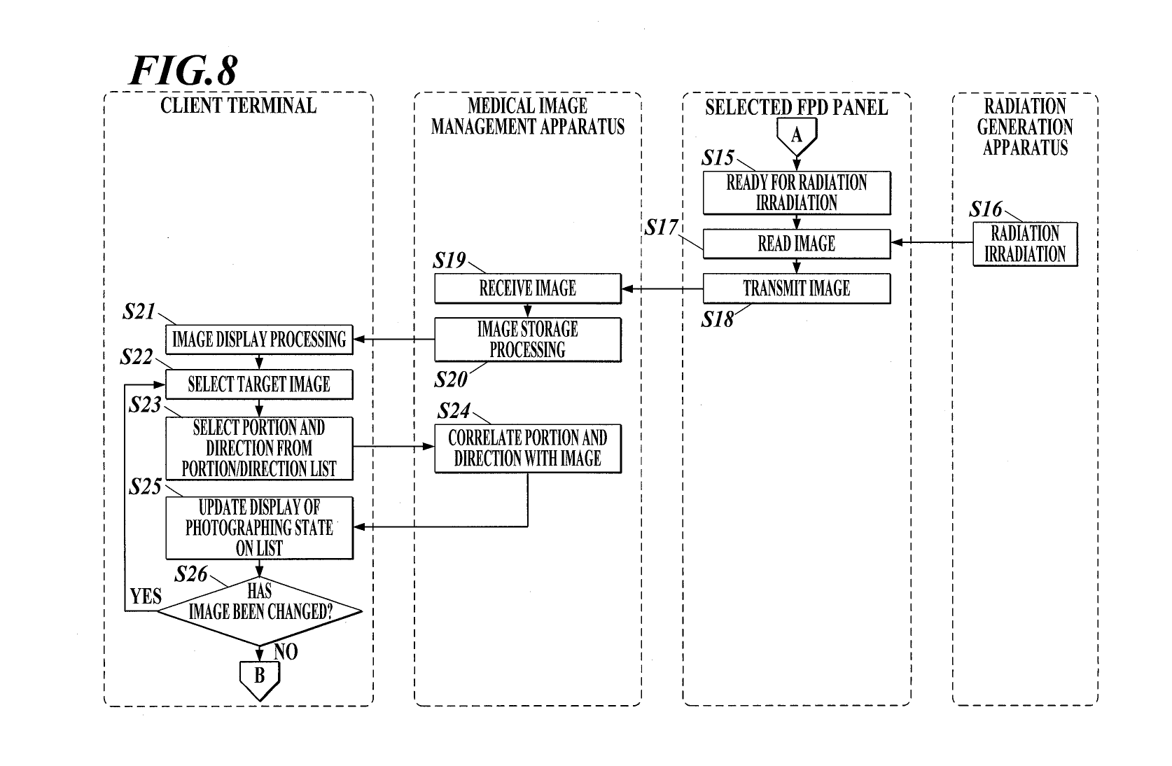

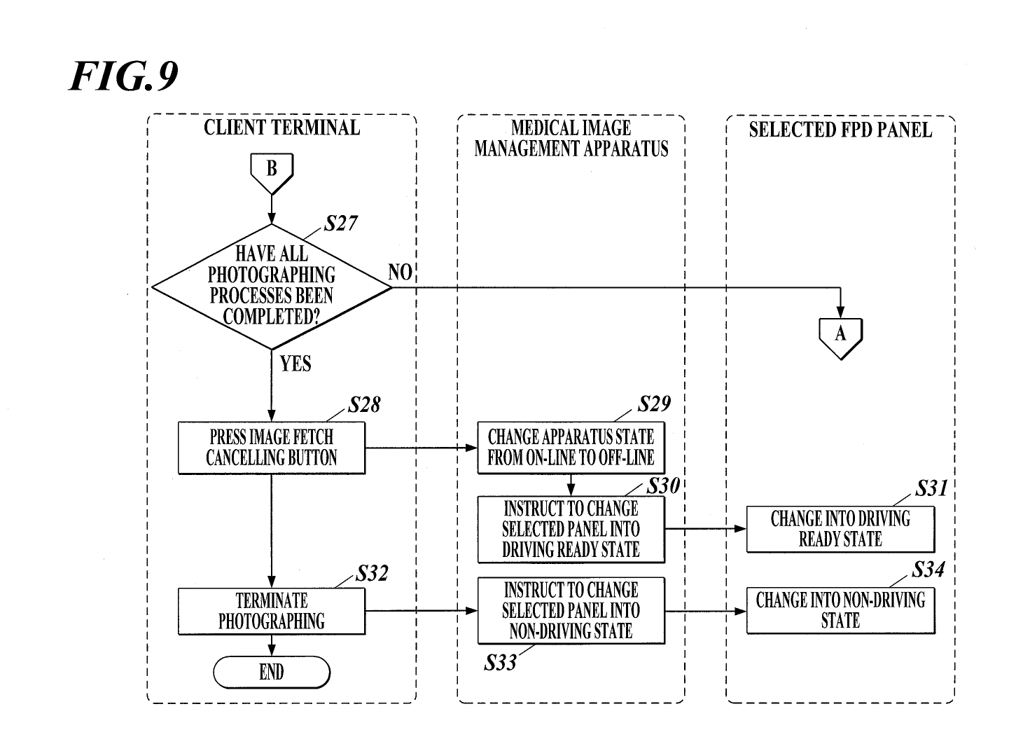

In the selected FPD panel 1 (the FPD panel 1 in the stable R1), when the wireless communication section 22 receives the driving instruction, the control section 19 changes the state of the FPD panel 1 to the driving state (Step S14). Then, the processing shifts to FIG. 8, and the selected FPD panel 1 is turned into the ready state for being irradiated with radiation from the radiation generation apparatus 50 (Step S15).

When the radiation generation apparatus 50 emits radiation (Step S16), in the selected FPD panel 1, the control section 19 detects the radiation irradiation by the radiation generation apparatus 50 to read out images (Step S17). Concretely, the control section 19 causes each radiation detection element 7 to start accumulation of electrical charge depending on the radiation irradiation, reads out the electrical charge accumulated in the radiation detection element 7 when a predetermined time has passed, and obtains image signals based on a dose of radiation, which has penetrated an object, to create image data.

Next, in the selected FPD panel 1, the control section 19 transmits the read medical image to the medical image management apparatus 30 via the wireless communication section 22 (Step S18).

In the medical image management apparatus 30, the communication section 34 receives the medical images transmitted from the selected FPD panel 1 (Step S19), and control section 31 causes the storage section 36 to store the received medical images (Step S20). Concretely, the control section 31 causes the storage section 36 to store the medical images fetched from the FPD panel 1, new records are added in the image information table T2 corresponding to the object examination for the photographing target horse, and the image IDs and the image file numbers of the fetched medical images are stored.

Then, in the medical image management apparatus 30, the control section 31 transmits the medical images received from the FPD panel 1, to the client terminal 40 which is set as the destination, via the communication section 34.

In the client terminal 40 which has transmitted the image fetching instruction, the communication section 44 receives the medical images transmitted from the medical image management apparatus 30, and the control section 41 causes the display section 43 to display the received medical images (Step S21).

For example, as illustrated in FIG. 10, the received medical image is displayed in the image display region A3 on the image display screen 431, and the thumbnail image D1 obtained by reducing the medical image displayed in the image display region A3 is displayed in the photographed image display region A5.

Next, in the client terminal 40, the target image is selected by operation using the operation section 42 (Step S22). Generally the client terminal 40 is in the state where the medical image which has been fetched from the photographing device such as the FPD panel 1 immediately before such operation and displayed in the image display region A3 is selected. In the case of changing the image to which the photographing portion and the photographing direction are correlated, the target image is selected among the thumbnail images displayed in the photographed image display region A5.

Then, when any one of the combinations (photographing object information) of the photographing portions and the photographing directions, which corresponds to the medical image currently displayed, is selected from the portion/direction list displayed in the display section 43 by operation using the operation section 42 (Step S23), the control section 41 transmits the selection information (portion/direction ID, etc.) indicating the selected combination of the photographing portion and the photographing direction to the medical image management apparatus 30 via the communication section 41.

For example, as the combination of the photographing portion and the photographing direction corresponding to the medical image displayed in the image display region A3, “right forefoot ankle joint: side face” C2 is selected from the portion/direction list displayed in the list display region A4 on the image display screen 431.

In the medical image management apparatus 30, the communication section 34 receives the selection information transmitted from the client terminal 40, and the control section 31 correlates the selected combination of the photographing portion and the photographing direction with the medical image generated by the FPD panel 1 on the basis of the selection information and stores them in the storage section 36 (Step S24). Concretely, the control section 31 causes the portion/direction ID corresponding to the selected combination of the photographing portion and the photographing direction to be stored, while being correlated with the image ID and the image file number of the target image, in the image information table T2 corresponding to the object examination for the photographing target horse.

Incidentally, it is possible to correlate the same portion/direction ID with the plural different medical images (image Ids and image file numbers) in the image information table T2.

Next, in the client terminal 40, the control section 41 updates the display of photographing state on the portion/direction list displayed in the display section 43 (Step S25). Specifically, the control section 41 causes the already-selected combination of the photographing portion and the photographing direction to be displayed distinguishably from other combinations of the photographing portions and the photographing directions on the portion/direction list on the basis of the image information table T2 stored in the storage section 36 of the medical image management apparatus 30.

More specifically, the control section 41 makes an inquiry to the medical image management apparatus 30, for each portion/direction displayed in the portion/direction list, whether or not the portion/direction ID corresponding to each portion/direction name in the photographing object information table T1 is included in the image information table T2 corresponding to the object examination for the photographing target horse.

In the medical image management apparatus 30, the control section 31 perform a search to confirm whether or not each portion/direction ID of the photographing object information table T1 is included in the image information table T2. When the portion/direction ID is contained in the image information table T2, it is determined that the portion/direction concerned has been correlated with the medical image, and the information indicating that the portion/direction has already been correlated with the medical image is transmitted to the client terminal 40 via the communication section 34. When the portion/direction ID is not contained in the image information table T2, the control section 31 determines that the portion/direction concerned has not been correlated with the medical image, and the information indicating that the portion/direction has not been correlated with the medical image is transmitted to the client terminal 40 via the communication section 34.

In the client terminal 40, the communication section 44 receives the information indicating whether or not the portion/direction has been correlated with the medical image. Then the control section 41 causes a check mark to be displayed, with regard to the combination of the photographing portion and the photographing direction which has already been correlated with the medical image, on the portion/direction list.

For example, in the photographing object information table T1 illustrated in FIG. 5, the portion/direction ID of “right forefoot ankle joint: side face” is “2”. The search is performed to confirm whether or not a record of “the portion/direction ID=2” is included in the image information table T2 corresponding to the object examination for the photographing target horse, and when the corresponding record exists, it is determined that “right forefoot ankle joint: side face” has been correlated with the medical image.

Incidentally, updating of display of the portion/direction list does not need to be performed for all combinations of the photographing portions and the photographing directions in the portion/direction list each time. It is also possible to perform such updating only for the combination of the photographing portion and the photographing direction selected in Step S23.

Also the configuration where the client terminal 40 holds the information indicating the combination of the photographing portion and the photographing direction selected in Step S23, and the selected combination of the photographing portion and the photographing direction is displayed, on the basis of this information, on the portion/direction list displayed in the display section 43, so that it is distinguishable from other combinations of the photographing portions and the photographing directions.

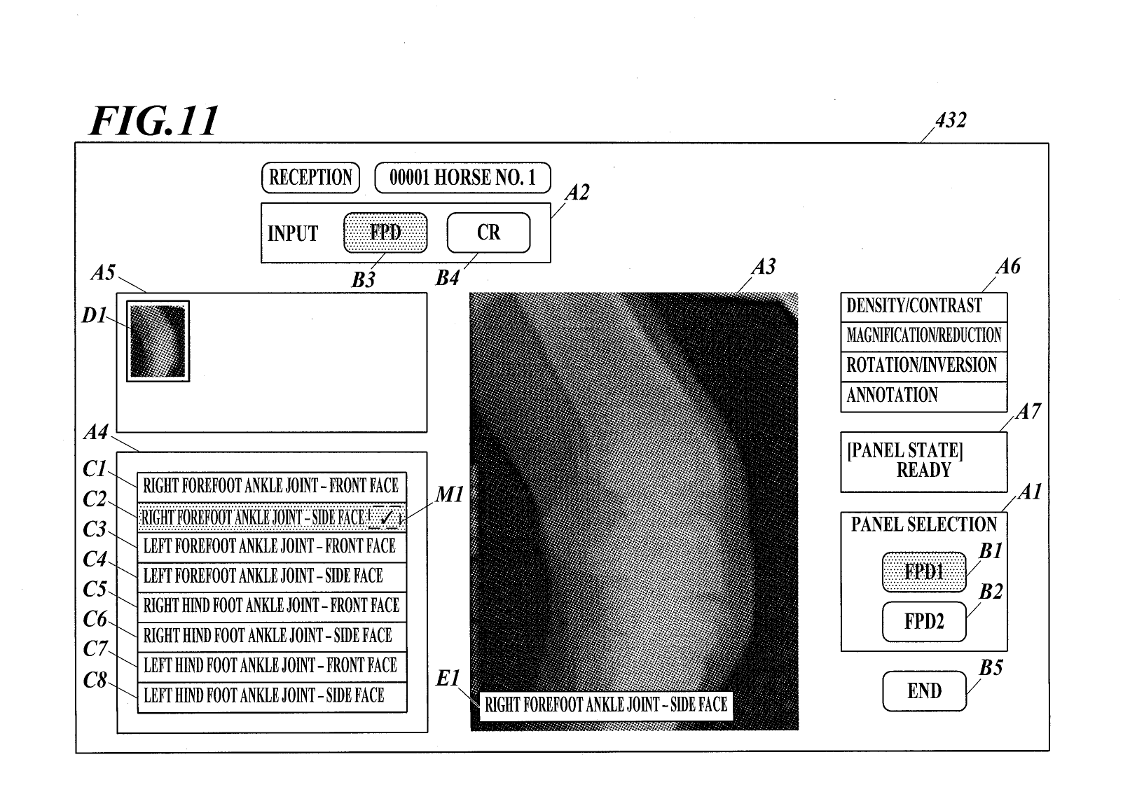

FIG. 11 illustrates an example of the image display screen 432 after the combination of the photographing portion and the photographing direction is correlated with the target image. As the regions A1 to A7 contained in the image display screen 432 are same as those of the image display screen 431, explanations thereof are omitted.

When “right forefoot ankle joint: side face” C2 is selected in the portion/direction list displayed in the list display region A4, a check mark M1 is added to the “right forefoot ankle joint: side face” C2 on the portion/direction list, and the portion/direction name E1 “right forefoot ankle joint: side face” is displayed on the medical image displayed in the image display region A3.

Then, when changing the target image (Step S26; YES), the target image is selected from among thumbnail images displayed in the photographed image display region A5, then the processing returns to Step S22, and the processes are repeated. Thus, it is also possible to change the combination of the photographing portion and the photographing direction to be correlated with the medical image, to which the combination of the photographing portion and the photographing direction has already been correlated.

When not changing the target image in Step S26 (Step S26; NO), the process shifts to FIG. 9. Then, a radiographer judges whether or not all photographing operations have been completed by confirming the portion/direction list displayed in the display section 43 (Step S27).

Here, when there are any process which has not been completed (Step S27; NO), the processing returns to Step S15, and the processes are repeated.

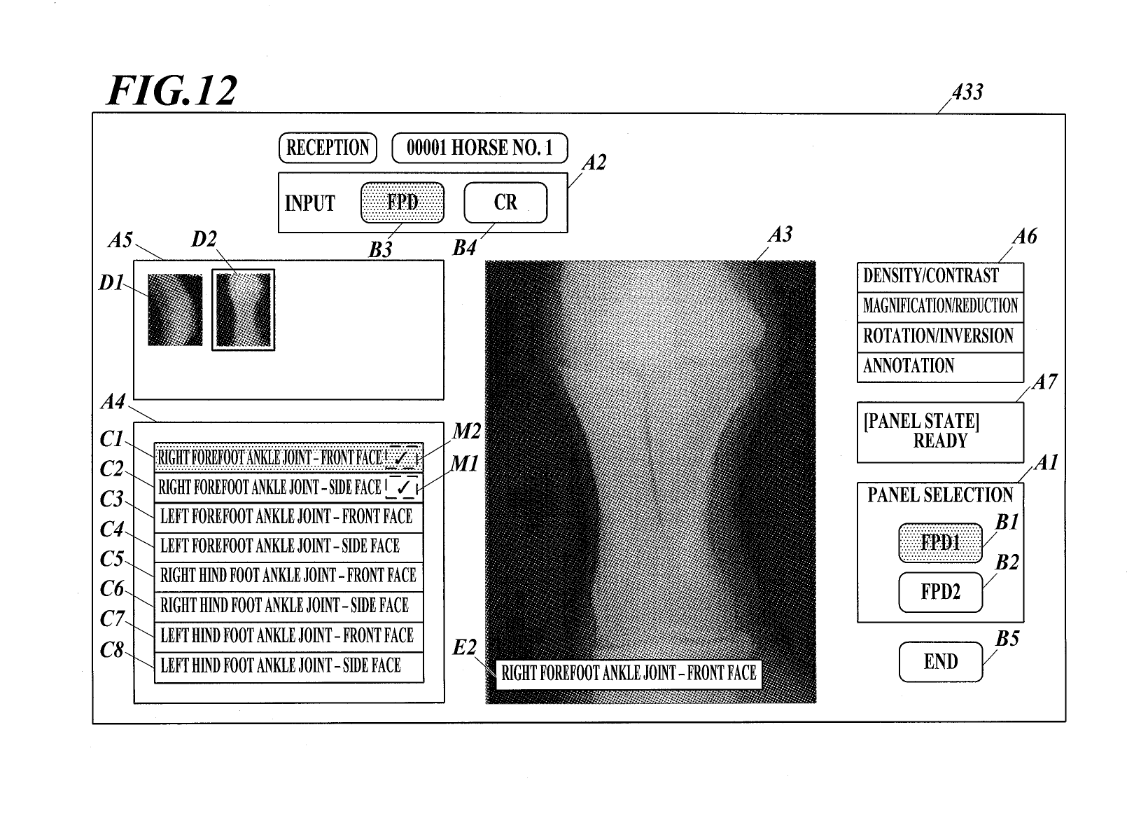

FIG. 12 illustrates an example of the image display screen 433 after photographing of “right forefoot ankle joint: front face” is performed subsequently to “right forefoot ankle joint: side face” and the combination of the photographing portion and the photographing direction is correlated with the target image. As the regions A1 to A7 contained in the image display screen 433 are same as those of the image display screen 431, explanations thereof are omitted.

The newly taken medical image is displayed in the image display region A3 on the image display screen 433, and the thumbnail image D2 obtained by reducing the medical image displayed in the image display region A3 is added in the photographed image display region A5.

When “right forefoot ankle joint: front face” C1 is selected in the portion/direction list displayed in the list display region A4, a check mark M2 is added to the “right forefoot ankle joint: front face” C1 on the portion/direction list, and the portion/direction name E2 “right forefoot ankle joint: front face” is displayed on the medical image displayed in the image display region A3.

Incidentally, it is possible to select the “right forefoot ankle joint: front face” C1 to which the check mark M1 has been added and/or the “right forefoot ankle joint: side face” C2 to which the check mark M2 has been added, again, as the combination of the photographing portion and the photographing direction corresponding to the medical image displayed in the image display region A3.

When all photographing operations have been completed in Step S27 (Step S27; YES), in the client terminal 40 installed in the stable R1, the image fetch cancelling button (the image fetch button B3 which is in a state of being pressed) is pressed on the image display screen by operation using the operation section 42 (Step S28). Then the control section 41 transmits the instruction to cancel the image fetch ready state to the medical image management apparatus 30 via the communication section 44.

In the medical image management apparatus 30, when the communication section 34 receives the instruction to cancel the image fetch ready state, the control section 31 changes the apparatus state from the on-line state to the off-line state (Step S29). Concretely, the control section 31 cancels the ready state for fetching the medical image created in the selected FPD panel 1.

Moreover, when the communication section 34 of the medical image management apparatus 30 receives the instruction to cancel the image fetch ready state, the control section 31 transmits the driving ready instruction that instructs the selected FPD panel 1 (the FPD panel 1 in the stable R1) to shift to the driving ready state via the communication section 34 (Step S30).

In the selected FPD panel 1 (the FPD panel 1 in the stable R1), when the wireless communication section 22 receives the driving ready instruction, the control section 19 changes the FPD panel 1 into the driving ready state (Step S31).

Next, in the client terminal 40 installed in the stable R1, when a completion button (see a completion button B5 illustrated in FIG. 10) on the image display screen is pressed by operation using the operation section 42, the control section 41 transmits a photographing completion instruction to the medical image management apparatus 30 via the communication section 44 (Step S32).

In the medical image management apparatus 30, when the communication section 34 receives the photographing completion instruction, the control section 31 transmits the non-driving instruction that instructs the selected FPD panel 1 to shift to the non-driving state via the communication section 34 (Step S33).

In the selected FPD panel 1, when the wireless communication section 22 receives the non-driving instruction, the control section 19 changes the FPD panel 1 into the non-driving state (Step S34).

With that, the medical image photographing processing terminates.

As described above, according to the first embodiment, the combinations of the photographing portions and the photographing directions to be used in photographing can be presented, by displaying the list of the combinations (photographing object information) of the photographing portions and the photographing directions which are contained in the examination. Moreover, by adding the check mark to the selected combination of the photographing portion and the photographing direction on the portion/direction list, the selected combination is displayed so that it can be distinguished from other combinations of the photographing portions and the photographing directions. This enables a user to easily recognize the combinations of the photographing portions and the photographing directions which have not been correlated to the medical images when performing photographing with respect to the plural combinations of the photographing portions and the photographing directions, which can prevent omission of photographing. Furthermore, because after photographing the combination of the photographing portion and the photographing direction is correlated with the medical image created by the photographing device such as the FPD panel 1, photographing can be easily performed in an arbitrary order when executing photographing of plural combinations of the photographing portions and the photographing directions.

There is a case that photographing is performed with respect to the same photographing portion and direction as a precautionary measure when a sufficient diagnosis capability cannot be obtained from one(1) medical image due to a pathology status. Heretofore, because a configuration where an image is correlated with a piece of photographing object information including a portion/direction information on a one-to-one basis has been adopted, it has been necessary to discard the image and execute photographing again, or to copy the photographing object information, to create the photographing object information regarding the same photographing portion and direction, and then perform photographing again.

In contrast, according to the first embodiment, because it is possible to correlate the same combination of the photographing portion and the photographing direction with the plural medical images created by the photographing device, operations for performing photographing again and/or performing a comprehensive diagnosis on the basis of the plural image, become easy. For example, it is possible to withdraw from a situation where a user notices that an original image is better after discarding the previously-taken image and performing photographing again, which has conventionally become a problem. The present invention is effective especially for animals because it is difficult to stop the movement of the photographing portion just as a radiographer intended and better image is not always obtained even when executing photographing again.

Incidentally, not only the check mark but also other marks may be added when displaying the selected photographing object information so that it is distinguishable from other photographing object information on the list of the photographing object information. Adding characters such as “already taken” to the selected photographing object information is also possible. Moreover, adding a background color to the selected photographing object information, and/or displaying the selected photographing object information with different background color from that of other photographing object information are also possible.

### Second Embodiment

Next, a second embodiment to which the present invention is applied will be described.

FIGS. 1 to 4 are employed herein because the medical image photographing system of the second embodiment has the similar configuration to that of the medical image photographing system 100 illustrated in the first embodiment, and an illustration and explanation of the configuration are omitted. Also the photographing object information table T1 (see FIG. 5), the image information table T2 (see FIG. 6), and the medical image photographing processing (see FIGS. 7 to 9) are similar to those of the first embodiment. Hereinafter, a configuration and processing specific to the second embodiment will be described.

The second embodiment is different from the first embodiment in a method for displaying the selected photographing object information so that it is distinguishable from other photographing object information on the portion/direction list displayed in the display section 43 of the client terminal 40.

The control section 41 causes the selected photographing object information to be displayed so that it is distinguishable from the other photographing object information by adding the medical image corresponding to the selected photographing object information on the portion/direction list displayed in the display section 43.

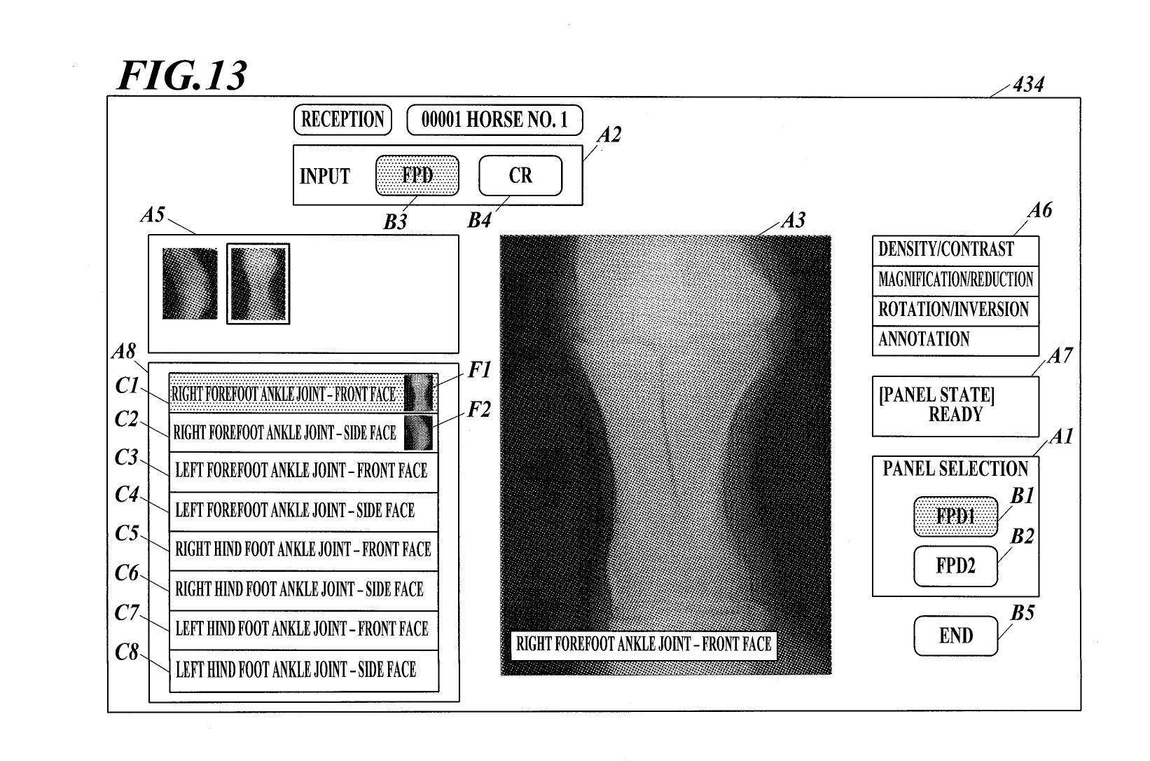

FIG. 13 illustrates an example of the image display screen 434 according to the second embodiment. Since the regions A1 to A3 and A5 to A7 contained in the image display screen 434 are same as those of the image display screen 431, explanations thereof are omitted.

A list display region A8 included in the image display screen 434 is a region in which the list of the photographing object information, namely, the list of combinations of the photographing portions and the photographing directions which are used in the examination to be executed is displayed. Incidentally, the list display region A8 is blank before the examination is selected.

With respect to the “right forefoot ankle joint: front face” C1 and the “right forefoot ankle joint: side face” C2 contained in the portion/direction list displayed in the list display region A8, the images F1, F2 corresponding to the combinations of the photographing portions and the photographing directions are displayed, respectively. With respect to the “left forefoot ankle joint: front face” C3, the “left forefoot ankle joint: side face” C4, the “right hind foot ankle joint: front face” C5, the “right hind foot ankle joint: side face” C6, the “left hind foot ankle joint: front face” C7 and the “left rear forefoot ankle joint: side face” C8, no image is displayed. As the images F1, F2, for example, the reduced images obtained by decreasing the number of pixels of the medical images corresponding to the combinations of the photographing portions and the photographing directions are used.

Incidentally, it is possible to select the “right forefoot ankle joint: front face” C1 to which the image F1 is added and/or the “right forefoot ankle joint: side face” C2 to which the image F2 is added, again, as the combination of the photographing portion and the photographing direction corresponding to the medical image displayed in the image display region A3, on the portion/direction list displayed in the list display region A8.

According to the second embodiment, in addition to the same advantages as those of the first embodiment, the combinations of the photographing portions and the photographing directions which have been selected on the portion/direction list can be displayed so that they are distinguishable from other combinations of the photographing portions and the photographing directions by adding the medical images corresponding to the selected combinations of the photographing portions and the photographing directions. By this, each correspondence relation between the combination of the photographing portion and direction and the medical image can be easily confirmed, which can prevents the correspondence relations from being mistakenly recognized.

### Third Embodiment

Next, a third embodiment to which the present invention is applied will be described.

FIGS. 1 to 4 are employed herein because the medical image photographing system of the third embodiment has the similar configuration to that of the medical image photographing system 100 illustrated in the first embodiment, and an illustrations and explanation of the configuration are omitted. Also the photographing object information table T1 (see FIG. 5), the image information table T2 (see FIG. 6), and the medical image photographing processing (see FIGS. 7 to 9) are similar to those of the first embodiment. Hereinafter, a configuration and processing specific to the third embodiment will be described.

The third embodiment is different from the first embodiment in a method for displaying the selected photographing object information so that it is distinguishable from other photographing object information on the portion/direction list displayed in the display section 43 of the client terminal 40.

The control section 41 causes the selected photographing object information to be displayed, on the portion/direction list displayed in the display section 43, so that it is distinguishable from other photographing object information, by changing a display position of the selected photographing object information into a different position from a display position of the other photographing object information.

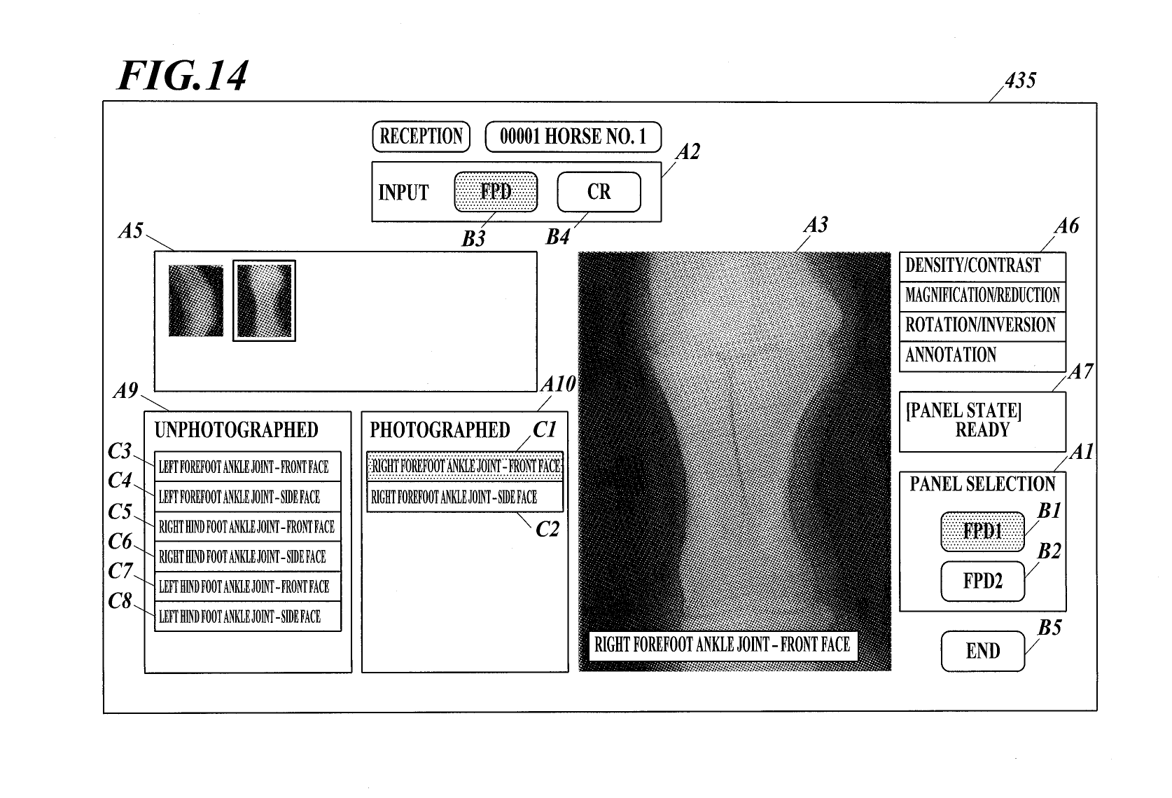

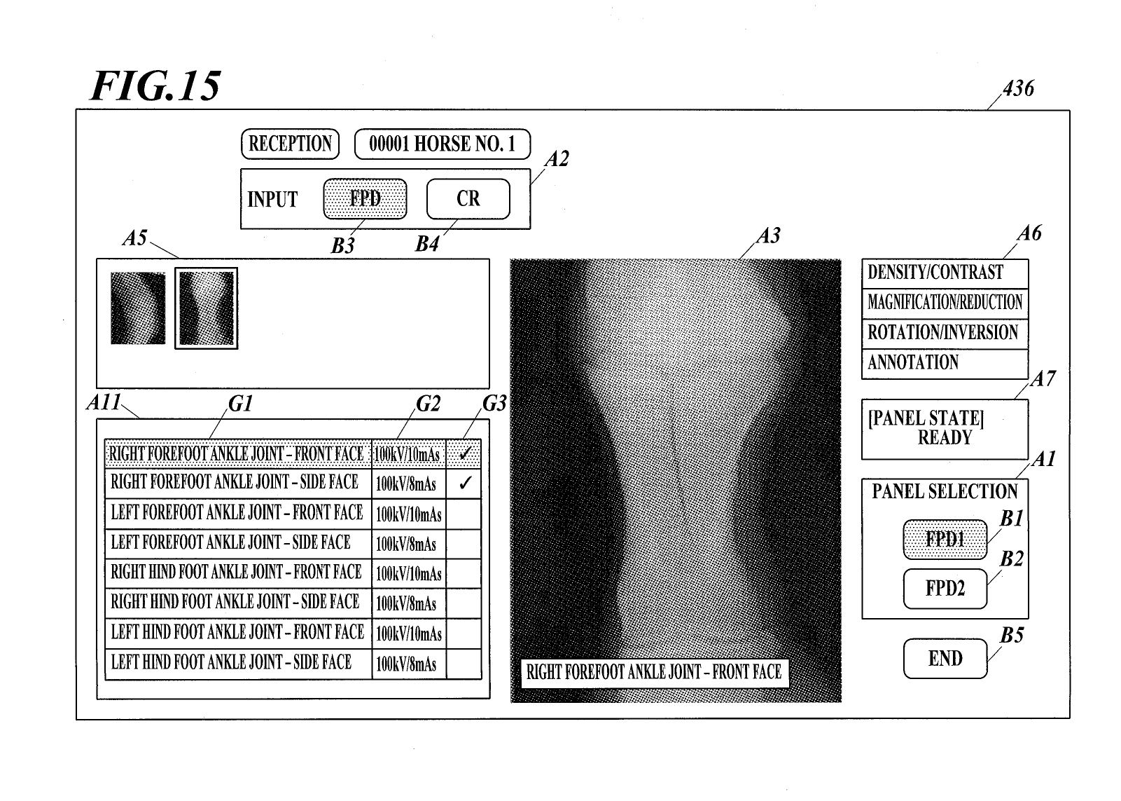

FIG. 14 illustrates an example of the image display screen 435 according to the third embodiment. As the regions A1 to A3 and A5 to A7 are similar to those of the image display screen 431, explanations thereof are omitted.

An unphotographed list display region A9 contained in the image display screen 435 is a region in which the combinations of the photographing portions and the photographing directions which have not been correlated with the medical image, among the listed combinations of the photographing portions and the photographing directions which are used in the examination to be conducted, are displayed. Incidentally, the unphotographed list display region A9 is blank before the examination is selected.

A photographed list display region A10 contained in the image display screen 435 is a region in which the combinations of the photographing portions and the photographing directions which have been correlated with the medical image are displayed. Incidentally, the photographed list display region A10 is blank before the combinations of the photographing portions and the photographing directions are selected with respect to the first sheet of the medical image.

The “right forefoot ankle joint: front face” C1 and the “right forefoot ankle joint: side face” C2 are displayed in the photographed list display region A10, and the “left forefoot ankle joint: front face” C3, the “left forefoot ankle joint: side face” C4, the “right hind foot ankle joint: front face” C5, the “right hind foot ankle joint: side face” C6, the “left hind foot ankle joint: front face” C7 and the “left rear forefoot ankle joint: side face” C8 are displayed in the unphotographed list display region A9. In other words, the “right forefoot ankle joint: front face” C1 and the “right forefoot ankle joint: side face” C2 are the selected combinations of the photographing portions and the photographing directions, and the “left forefoot ankle joint: front face” C3, the “left forefoot ankle joint: side face” C4, the “right hind foot ankle joint: front face” C5, the “right hind foot ankle joint: side face” C6, the “left hind foot ankle joint: front face” C7 and the “left rear forefoot ankle joint: side face” C8 are the unselected combinations of the photographing portions and the photographing directions.

Incidentally, it is possible to select the “right forefoot ankle joint: front face” C1 and/or the “right forefoot ankle joint: side face” C2 which are displayed in the photographed list display region A10, again, as the combination of the photographing portion and the photographing direction corresponding to the medical image displayed in the image display region A3.

According to the third embodiment, in addition to the same advantages as that of the first embodiment, the selected combinations of the photographing portions and the photographing directions can be displayed, on the portion/direction list, so that they are distinguishable from other combinations of the photographing portions and the photographing directions, by changing the display position of the selected combinations into the different position from the display position of the unselected combinations. By this, it becomes possible to easily recognize whether or not each combination of the photographing portion and the photographing direction has been correlated to the medical image, which can prevent omission of photographing.

### Fourth Embodiment

Next, a fourth embodiment to which the present invention is applied will be described.

FIGS. 1 to 4 are employed herein because the medical image photographing system of the fourth embodiment has the similar configuration to that of the medical image photographing system 100 illustrated in the first embodiment, and an illustrations and explanation of the configuration are omitted. Also the image information table T2 (see FIG. 6) and the medical image photographing processing (see FIGS. 7 to 9) are similar to those of the first embodiment. Hereinafter, a configuration and processing specific to the fourth embodiment will be described.

The fourth embodiment is different from the first embodiment in the point that also photographing conditions are displayed when displaying the portion/direction list in the display section 43 of the client terminal 40.

In the fourth embodiment, in the photographing object information table T1 illustrated in FIG. 5, also each photographing condition corresponding to each piece of the photographing object information is displayed, for each piece of the photographing object information.

The control section 41 causes each photographing condition corresponding to each piece of photographing object information, in addition to each piece of photographing object information (each combination of the photographing portion and the photographing direction), on the portion/direction list displayed in the display section 43, on the basis of the photographing object information table T1.

FIG. 15 illustrates an example of the image display screen 436 according to the fourth embodiment. As the regions A1 to A3 and A5 to A7 contained in the image display screen 436 are same as those of the image display screen 431, explanations thereof are omitted.

A list display region A11 included in the image display screen 436 is a region in which a list of the combinations of the photographing portions and the photographing directions which are used in the examination to be executed is displayed. Incidentally, the list display region 11 is blank before the examination is selected.

The list display region 11 includes a portion/direction display column G1, a photographing condition display column G2, and a check mark display column G3.

In the portion/direction display column G1, the photographing object information, namely, the combinations of the photographing portions and the photographing directions are displayed.

In the photographing condition display column G2, the photographing conditions respectively corresponding to the combinations of the photographing portions and the photographing directions displayed in the portion/direction display column G1 are displayed. Here, an X-ray tube voltage [kV] and a radiation exposure dose [mAs] are displayed as each of the photographing conditions.

In the check mark display column G3, a check mark is displayed with respect to each combination of the photographing portion and the photographing direction which is displayed in the portion/direction display column G1 and has been correlated to the medical image.

According to the fourth embodiment, in addition to the same advantages as those of the first embodiment, a radiographer can easily know the proper photographing condition for each photographing portion/direction because the photographing conditions respectively corresponding to the combinations of the photographing portions and the photographing directions are displayed on the portion/direction list, in addition to the combinations of the photographing portions and the photographing directions.

### Fifth Embodiment

Next, a fifth embodiment to which the present invention is applied will be described.

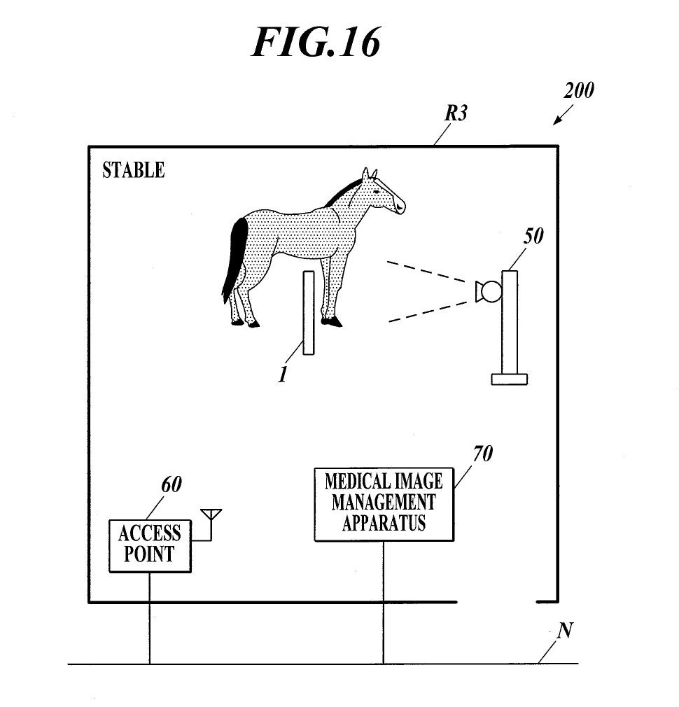

FIG. 16 illustrates a system configuration of a medical image photographing system 200 according to the fifth embodiment. The medical image photographing system 200 is configured to include the FPD panel 1, a medical image management apparatus 70, the radiation generation apparatus 50, the access point 60, and so on. The FPD panel 1 and the medical image management apparatus 70 are connected to each other so that data communication can be performed therebetween via the access point 60 and the communication network N. In a stable R3, radiation images of racehorses as photographing objects are taken.

Since the FPD panel 1, the radiation generation apparatus 50, and the access point 60 are already described in the first embodiment, explanations thereof are omitted.

The medical image management apparatus 70 is a computer apparatus which has both functions of the medical image management apparatus 30 and the client terminal 40 of the first embodiment, and manages the medical images created in the photographing device such as the FPD panel 1.

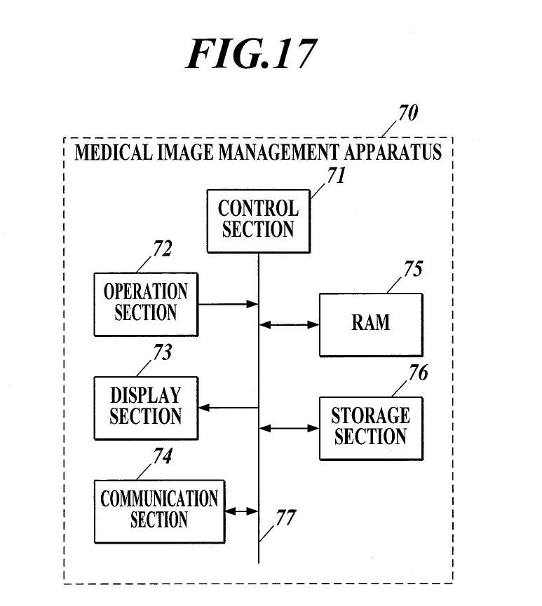

FIG. 17 illustrates a functional configuration of the medical image management apparatus 70.

As illustrated in FIG. 17, the medical image management apparatus 70 is configured to include a control section 71, an operation section 72, a display section 73, a communication section 74, a RAM 75, a storage section 76, and so on, which are connected to one another by a bus 77.

The control section 71 is composed of a CPU and the like, reads out various programs such as system programs and processing programs stored in the storage section 76 to expand them in the RAM 75, and executes various processes according to the expanded programs.

The operation section 72 is configured to include a keyboard equipped with character input keys, numeric input keys, various functional keys, etc., a pointing device such as a mouse, and so on, and outputs a pressing signal of each key which has been pressed on the keyboard and an operation signal from the mouse to the control section 71 as input signals.

The operation section 72 is used when selecting any one piece of the photographing object information, with respect to the medical image generated by the photographing device such as the FPD panel 1, from the list displayed in the display section 73. Thus, the operation section 72 functions as a selection section.

Also this embodiment describes the case that the photographing object information includes the photographing portion and the photographing direction, the photographing object information may include a body posture.

The display section 73 is equipped with a monitor such as an LCD to display various screens according to instructions of display signals input from the control section 71.

The communication section 74 is composed of a network interface and the like, and performs data transmission/reception with an external device/apparatus connected to the communication network N.

The RAM 75 functions as a work area in which various programs read out from the storage section 76 and executable by the control section 71, input/output data, etc. are temporarily stored in various processes executed and controlled by the control section 71.

The storage section 76 is composed of an HDD, a semiconductor nonvolatile memory, or the like. The storage section 76 stores various programs and data necessary for various processes. For example, the storage section 76 stores the photographing object information table T1 (see FIG. 5) and the image information table T2 (see FIG. 6).

Since the photographing object information table T1 and the image information table T2 are already described in the first embodiment, explanations thereof are omitted.

The control section 71 causes the display section 73 to display a list (portion/direction list) of pieces of photographing object information contained in the photographing object information table T1 on the basis of the photographing object information table T1 including the plural pieces of photographing object information. Thus, the control section 71 functions as a display control section.

The control section 71 correlates the selected photographing object information with the medical image created by the photographing device such as the FPD panel 1 and causes the storage section 76 to store them. Concretely, the control section 71 correlates the portion/direction ID corresponding to the selected photographing object information with the image ID and the image file number corresponding to the medical image in the image information table T2. Thus, the control section 71 functions as a correlation section.

The control section 71 may correlates the same photographing object information with the plural medical images created by the photographing device such as the FPD panel 1.

The control section 71 causes the selected photographing object information to be displayed so that it is distinguishable from other photographing object information on the portion/direction list displayed in the display section 73 on the basis of the image information table T2 stored in the storage section 76.

For example, the control section 71 causes the selected photographing object information to be displayed so that it is distinguishable from other photographing object information, by adding a mark, characters, background color, or the medical image corresponding to the selected photographing object information, on the portion/direction list displayed in the display section 73 (see FIGS. 12, 13).

Alternatively, the control section 71 causes the selected photographing object information to be displayed so that it is distinguishable from other photographing object information by changing a display position of the selected photographing object information into a different position from a display position of other photographing object information (see FIG. 14).

Moreover, the control section 71 may causes the photographing conditions respectively corresponding to pieces of the photographing object information to be displayed, in addition to the photographing object information, on the portion/direction list displayed in the display section 73 on the basis of the photographing object information table T1 (see FIG. 15).

Next, an operation of the medical image management apparatus 70 will be described.

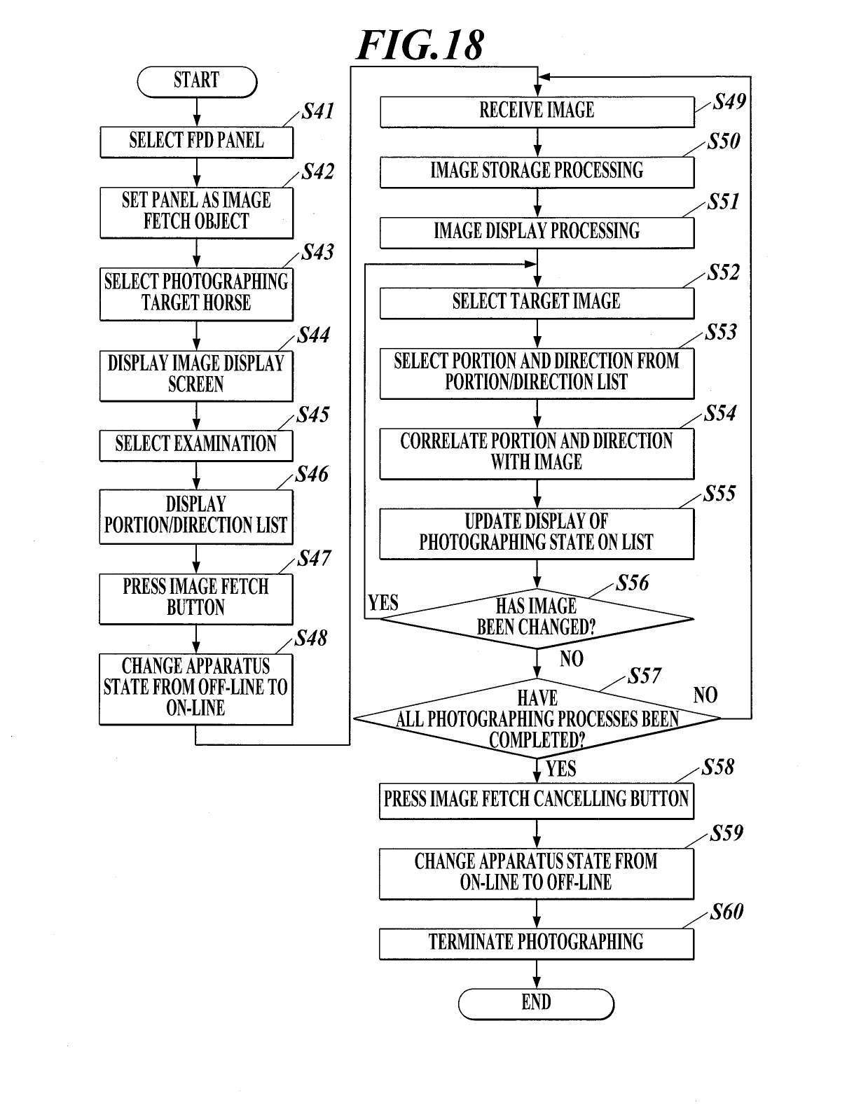

FIG. 18 is a flowchart illustrating processing in the medical image management apparatus 70.

First, in the medical image management apparatus 70 installed in the stable R3, the control section 71 reads out the information indicating each FPD panel 1 in the medical image photographing system 200 and the previously registered information regarding the horses, and causes the display section 73 to display the reception screen on the basis of the read out information. The configuration of the reception screen is similar to that of the first embodiment.

When any one of panel selection buttons in the panel selection region is pressed by operation using the operation section 72 and the FPD panel 1 in the stable R3 to be the image fetch target is selected (Step S41), the control section 71 sets the selected FPD panel 1 to the FPD panel 1 as the image fetch target (Step S42).

Next, when the photographing target horse is selected from among the horses displayed in the horse list display region on the reception screen by operation using the operation section 72 (Step S43), the control section 71 causes the display section 73 to display the image display screen for displaying the image of the selected photographing target horse (Step S44). The configuration of the image display screen is similar to that of the first embodiment (see FIGS. 10 to 12).

In the medical image management apparatus 70, the control section 71 transmits the non-driving instruction that instructs the non-selected FPD panel 1, among the plural FPD panels 1 existing, to shift to the non-driving state at the timing when the screen displayed in the display section 73 changes into the image display screen of the photographing target horse, via the communication section 74, and transmits the driving ready instruction that instructs the selected FPD panel 1 to shift to the driving ready state via the communication section 74.

In the non-selected FPD panel 1, when the wireless communication section 22 receives the non-driving instruction, the control section 19 changes the non-selected FPD panel 1 into the non-driving state.

In the selected FPD panel 1, when the wireless communication section 22 receives the driving ready instruction, the control section 19 changes the selected FPD panel 1 into the driving ready state.

Then, in the medical image management apparatus 70, when the examination to be executed this time is selected from among the plural examinations previously registered as the racehorse examinations by operation using the operation section 72 (Step S45), the control section 71 reads out the photographing object information table T1 corresponding to the selected examination from the storage section 76. Then, the control section 71 causes the display section 73 to display the list (portion/direction list) of the combinations of the photographing portions and the photographing directions to be used in the selected examination on the basis of the plural pieces of the photographing object information contained in the read out photographing object information table T1 (Step S46).

When the image fetch button is pressed on the image display screen by operation using the operation section 72 (Step S47), the control section 71 changes the apparatus state from off-line to on-line (Step S48). Concretely, the control section 71 changes the apparatus into the ready state for fetching the medical images generated in the selected FPD panel 1.

Moreover, when the image fetch button is pressed in the medical image management apparatus 70, the control section 71 transmits the driving instruction that instructs the selected FPD 1 to shift to the driving state via the communication section 74.

In the selected FPD panel 1, when the wireless communication section 22 receives the driving instruction, the control section 19 changes the selected FPD panel into the driving state so that it is in the ready state for being irradiated with radiation from the radiation generation apparatus 50.

When the radiation generation apparatus 50 emits irradiation, the control section 19 of the selected FPD panel 1 detects radiation irradiation by the radiation generation apparatus 50 to read the image. The control section 19 then transmits the read medical image to the medical image management apparatus 70 via the wireless communication section 22.

In the medical image management apparatus 70, the communication section 74 receives the medical image transmitted from the selected FPD panel 1 (Step S49), and the control section 71 causes the storage section 76 to store the received medical image (Step S50). Specifically, the control section 71 causes the storage section 76 to store the medical image fetched from the FPD panel 1, and at the same time, new record is added to the image information table T2 corresponding to the object examination for the photographing target horse and the image ID and the image file number of the fetched medical image are stored in the storage section 76.

Next, the control section 71 causes the display section 73 to display the medical image received from the FPD panel 1 (Step S51).

Then, the target image is selected by operation using the operation section (Step S52). Generally the medical image which has been fetched from the photographing device such as the FPD panel 1 immediately before the operation and displayed in the image display region A3 (see FIG. 10) is selected. In the case of changing the image to which the photographing portion and the photographing direction are correlated, the target image is selected from among the thumbnail images displayed in the photographed image display region A5 (see FIG. 10).

When any one of the combinations (photographing object information) of the photographing portions and the photographing directions corresponding to the currently-displayed medical image is selected from the portion/direction list displayed in the display section 73 (Step S53) by operation using the operation section 72, the control section 71 correlates the selected combination of the photographing portion and the photographing direction with the medical image created by the FPD panel 1 and causes the storage section 76 to store them (Step S54). Concretely, the control section 71 causes the portion/direction ID corresponding to the selected combination of the photographing portion and the photographing direction to be stored while correlating it with the image ID and the image file number of the target image in the image information table T2 corresponding to the object examination for the photographing target horse.

Incidentally, it is possible to correlate the same portion/direction ID with the plural different medical images (image IDs and image file numbers) in the image information table T2.

Next, the control section 71 updates the display of the photographing state on the portion/direction list displayed in the display section 73 (Step S55). Concretely, the control section 71 causes the already-selected combination of the photographing portion and the photographing direction to be displayed so that it is distinguishable from other combinations of the photographing portions and the photographing directions on the portion/direction list on the basis of the image information table T2 stored in the storage section 76.

More concretely, the control section 71 performs a search, with respect to each portion/direction displayed in the portion/direction list, to confirm whether or not the portion/direction ID corresponding to each portion/direction name of the photographing object information table T1 is included in the image information table T2 corresponding to the object examination for the photographing target horse. When the portion/direction ID is contained in the image information table T2, it is determined that the portion/direction has been correlated with the medical image, and when the portion/direction ID is not contained in the image information table T2, it is determined that the portion/direction has not been correlated with the medical image.

The method for displaying the already-selected photographing object information so that it is distinguishable from other photographing object information on the portion/direction list is not limited to the examples illustrated in the first to fourth embodiments, and any other methods may be adopted.

Incidentally, updating of display of the portion/direction list does not need to be performed for all combinations of the photographing portion and the photographing direction in the portion/direction list each time. It is also possible to perform such updating only for the combination of the photographing portion and the photographing direction selected in Step S53.

Next, when changing the target image (Step S56; YES), the target image is selected from among the thumbnail images displayed in the photographed image display region A5 (see FIG. 10) by operation using the operation section 72, then the processing returns to Step S52, and the processes are repeated.

When not changing the target image in Step S56 (Step S56; NO), a radiographer judges whether or not all photographing operations are completed by confirming the portion/direction list displayed in the display section 73 (Step S57).

Here, if there is a not-completed photographing operation (Step S57; NO), the processing returns to Step S49 and the processes are repeated.

When all photographing operations are completed in Step S57 (Step S57; YES), the image fetch cancelling button is pressed on the image display screen by operation using the operation section 72 (Step S58), and the control section 71 changes the apparatus state from on-line to off-line (Step S59). Specifically, the control section 71 cancels the ready state for fetching the medical image created in the selected FPD panel 1.

Moreover, when the image fetch cancelling button is pressed, the control section 71 transmits the driving ready instruction that instructs the selected FPD panel 1 to shift to the driving ready state via the communication section 74.

In the selected FPD panel 1, when the wireless communication section 22 receives the driving ready instruction, the control section 19 changes the selected FPD panel 1 into the driving ready state.

Then, in the medical image management apparatus 70, when the completion button on the image display screen is pressed by operation of the operation section 72 (Step S60), the control section 71 transmits the non-driving instruction that instructs the selected FPD panel 1 to shift to the non-driving state via the communication section 74.

In the selected FPD panel 1, when wireless communication section 22 receives the non-driving instruction, the control section 19 changes the selected FPD panel 1 into the non-driving state.

With that, the processing in the medical image management apparatus terminates.

As described above, according to the fifth embodiment, the combinations of the photographing portions and the photographing directions to be photographed can be presented by displaying the list of the combinations (photographing object information) of the photographing portions and the photographing directions included in the examination. Moreover, because the selected combinations of the photographing portions and the photographing directions are displayed so that they can be distinguished from other combinations of the photographing portions and the photographing directions on the portion/direction list, it becomes easy to recognize the combinations of the photographing portions and the photographing directions which have not been correlated with the medical images when photographing the plural combinations of the photographing portions and the photographing directions, and thereby photographing in an arbitrary order becomes easier.

Moreover, according to the fifth embodiment, since the same combination of the photographing portion and the photographing direction can be correlated with the plural medical images created by the photographing device, operations for executing photographing again and/or performing a comprehensive diagnosis on the basis of the plural images become easier.

Incidentally, the descriptions of the above embodiments are mere examples of the medical image photographing system and medical image management apparatus according to the present invention, and the present invention is not limited thereto. The detailed configurations and detailed operations of the devices/apparatuses constituting the system can be arbitrarily changed without departing from the spirit of the present invention.

For example, the fourth embodiment is the example of the case where the photographing conditions are displayed on the list of the photographing object information similar to that of the first embodiment, but it is also possible to display the photographing conditions on the list of the photographing object information similar to the second embodiment or the third embodiment. Moreover, the photographing conditions to be displayed may be X-ray tube current [mA] and exposure time [msec] in the radiation generation apparatus 50, etc.

Although the cases of using the combinations of the photographing portions and the photographing directions as the photographing object information are described in the above embodiments, it is also possible to display body postures, in addition to the combinations of the photographing portions and the photographing directions, on the list of the photographing object information, when the photographing object information contains the body postures. In the case that the body posture to be generally photographed is already determined with respect to each combination of the photographing portion and the photographing direction, the display of the body postures may be omitted. Furthermore, in the case that the general photographing direction is already determined with respect to the photographing portion, the photographing object information may not include the photographing directions.

Also a configuration where the pieces of photographing object information are automatically selected in a predetermined order on the list of the photographing object information and the selection is confirmed when an enter button is pressed may be adopted. For example, among the pieces of photographing object information which have not been correlated with the medical images, the first one in a predetermined order and/or the secondary one to the precedingly selected photographing object information is automatically selected. Even in such case, the photographing object information to be correlated with the medical image may be changed by operation using the operation section 42 of the client terminal 40 or the operation section 72 of the medical image management apparatus 70.

Concretely, there is simulated the case that, when photographing the above described photographing portions/directions (1)-(8), photographing object is hurriedly changed from “right forefoot ankle joint: front face” (1) to “right hind foot ankle joint: front face” (5) because a horse begins to move its forefoot, photography of (6) to (8) is performed in order subsequently to photography of (5), then processing returns to photography of “right forefoot ankle joint: side face” (2), and also photography of (3) and (4) is performed. In this case, if the photographing order is previously set to the order of (1) to (8), a manual operation to be executed by the radiographer is to select the combination two times, namely, select the combinations of the photographing portions/directions (5) and (2). Other combinations can be selected by pressing the enter button while the combination subsequent to the precedingly selected combination is sequentially selected.

Moreover, though the cases of using the FPD panel as the photographing device are described in the above embodiments, a Computed Radiography (CR) device, an Ultrasound diagnosis apparatus (US), etc. may be used as the photographing device.

Furthermore, though the cases that a horse is the photographing object are described in the above embodiments, the photographing object may be a human being or animals other than the horse.

It is also possible to display the fact that a plurality of medical images exists and/or the number of the medical images on the list of the photographing object information, when there are plural medical images to which the same photographing object information has been correlated.

The present U.S. patent application claims a priority under the Paris Convention of Japanese patent application No. 2013-019196 filed on Feb. 4, 2013, in which all contents of this application are disclosed, and which shall be a basis of correction of an incorrect translation.

What is claimed is:

1. A medical image photographing system comprising:
a photographing device that generates at least one medical image of a photographing object;
a medical image management apparatus that is connected to the photographing device so as to enable data communication with the photographing device and manages the medical image generated by the photographing device; and
a client terminal that is connected to the medical image management apparatus so as to enable data communication with the medical image management apparatus, wherein
the client terminal includes:
a display section:
a display control section to cause the display section to display a list of a plurality of pieces of photographing object information on the basis of the photographing object information including at least a photographing portion;
a selection section to receive an input for selecting any one piece of photographing object information, from the list displayed in the display section, with respect to the medical image generated by the photographing device; and
a transmission section to transmit selection information indicating the selected photographing object information to the medical image management apparatus, and
the medical image management apparatus includes:
a storage section; and
a correlation section to correlate the selected photographing object information with the medical image generated by the photographing device on the basis of the selection information transmitted from the client terminal to cause the storage section to store the selected photographing object information correlated to the medical image, and
the display control section causes the selected photographing object information to be displayed, on the list displayed in the display section, so that the selected photographing object information is distinguishable from other photographing object information.

2. The medical image photographing system of claim 1, wherein the correlation section is capable of correlating the same photographing object information with the plurality of medical images generated by the photographing device.

3. The medical image photographing system of claim 1, wherein the display control section causes the selected photographing object information to be displayed, on the list displayed in the display section, so that the selected photographing object information is distinguishable from other photographing object information, by adding a mark, a character, a background color, or the medical image corresponding to the selected object information.

4. The medical image photographing system of claim 1, wherein the display control section causes the selected photographing object information to be displayed, on the list displayed in the display section, so that the selected photographing object information is distinguishable from other photographing object information, by changing a display position of the selected photographing object information into a different position from a display position of the other photographing object information.

5. The medical image photographing system of claim 1, wherein the photographing object information further includes a photographing direction or a body posture.

6. The medical image photographing system of claim 1, wherein the display control section causes a photographing condition corresponding to each piece of the photographing object information to be displayed, on the list displayed in the display section, in addition to each piece of the photographing object information.

7. A medical image management apparatus which is connected to a photographing device that generates at least one medical image of a photographing object so as to enable data communication with the photographing device, and manages the medical image generated by the photographing device, the medical image management apparatus comprising:
a display section:
a display control section to cause the display section to display a list of a plurality of pieces of photographing object information on the basis of the photographing object information including at least a photographing portion;
a selection section to receive an input for selecting any one piece of photographing object information, from the list displayed in the display section, with respect to the medical image generated by the photographing device;
a storage section; and
a correlation section to correlate the selected photographing object information with the medical image generated by the photographing device to store the selected photographing object information correlated to the medical image, wherein
the display control section causes the selected photographing object information to be displayed, on the list displayed in the display section, so that the selected photographing object information is distinguishable from other photographing object information.

8. The medical image management apparatus of claim 7, wherein the correlation section is capable of correlating the same photographing object information with the plurality of medical images generated by the photographing device.

9. The medical image management apparatus of claim 7, wherein the display control section causes the selected photographing object information to be displayed, on the list displayed in the display section, so that the selected photographing object information is distinguishable from other photographing object information, by adding a mark, a character, a background color, or the medical image corresponding to the selected object information.

10. The medical image management apparatus of claim 7, wherein the display control section causes the selected photographing object information to be displayed, on the list displayed in the display section, so that the selected photographing object information is distinguishable from other photographing object information, by changing a display position of the selected photographing object information into a different position from a display position of the other photographing object information.

11. The medical image management apparatus of claim 7, wherein the photographing object information further includes a photographing direction or a body posture.

12. The medical image management apparatus of claim 7, wherein the display control section causes a photographing condition corresponding to each piece of the photographing object information to be displayed, on the list displayed in the display section, in addition to each piece of the photographing object information.
